(19) United States
(12) Patent Application Publication
(10) Pub. No.: US 2002/0111472 A1
(43) Pub. Date: Aug. 15, 2002

(54) T1-R LIGAND III

(76) Inventors: JIAN NI, ROCKVILLE, MD (US); REINER GENTZ, SILVER SPRING, MD (US); STEVEN M. RUBEN, OLNEY, MD (US)

Correspondence Address: HUMAN GENOME SCIENCES INC, 9410 KEY WEST AVENUE, ROCKVILLE, MD, 20850

(21) Appl. No.: 09/030,847

(22) Filed: Feb. 26, 1998

Related U.S. Application Data
(60) Provisional application No. 60/039,483, filed on Feb. 28, 1997.

(51) Int. Cl. C07H 21/04; C12N 15/00; C12N 15/09; C07K 7/00; C12N 15/63; C12N 15/70; C12N 15/74; C07K 5/00; C07K 16/00; C07K 17/00; A61K 38/00; C07K 1/00; C07K 14/00; C12P 21/08; C12N 15/85; C12N 15/87

(52) U.S. Cl. 536/23.5; 435/455; 435/320.1; 530/324; 530/387.7

(57) ABSTRACT

The present invention relates to a novel T1-R ligand III protein which is a member of the IL-1RI ligand-like family. In particular, isolated nucleic acid molecules are provided encoding the human T1-R ligand III protein. T1-R ligand III polypeptides are also provided as are vectors, host cells and recombinant methods for producing the same. The invention further relates to screening methods for identifying agonists and antagonists of T1-R ligand III activity. Also provided are diagnostic methods for detecting immune system-related disorders and therapeutic methods for treating immune system-related disorders.

This application claims benefit of 35 U.S.C. section 119(e) based on copending U.S. Provisional Application Serial No: 60/039,483, filed Feb. 28, 1997.

### FIELD OF THE INVENTION

The present invention relates to two splice variants of a novel human gene, each of which encodes a separate and independent polypeptide which is a member of the T1-R family. More specifically, isolated nucleic acid molecules are provided which encode two splice variants of a human polypeptide named T1-R ligand III. T1-R ligand III polypeptides are also provided, as are vectors, host cells and recombinant methods for producing the same. Also provided are diagnostic methods for detecting disorders related to the immune system, and therapeutic methods for treating such disorders.

The invention further relates to screening methods for identifying agonists and antagonists of T1-R ligand III activity.

### BACKGROUND OF THE INVENTION

Related Art

Interleukin-1 (IL-1). Interleukin-1 (IL-1α and IL-1β) is a “multifunctional” cytokine that affects nearly every cell type, and often in concert with other cytokines or small mediator molecules (Dinarello, C. A., Blood 87:2095-2147 (Mar. 15, 1996)). There are three members of the IL-1 gene family: IL-1α, IL-1β, and IL-1 receptor antagonist (IL-1Ra). IL-1α and IL-1β are agonists and IL-1Ra is a specific receptor antagonist. IL-1α and β are synthesized as precursors without leader sequences. The molecular weight of each precursor is 31 kD. Processing of IL-1α and IL-1β to “mature” forms of 17 kD requires specific cellular proteases. In contrast, IL-1Ra evolved with a signal peptide and is readily transported out of the cells and termed secreted IL-1Ra (sIL-1Ra).

IL-1 Receptor and Ligands.

The receptors and ligands of the IL-1 pathway have been well defined (for review, see Dinarello, C. A., FASEB J. 8:1314-1325 (1994); Sims, J. E., et al., Interleukin-1 signal transduction: Advances in Cell and Molecular Biology of Membranes and Organelles, Vol. 3, JAI Press, Inc., Greenwich, Conn. (1994), pp. 197-222). Three ligands, IL-1α, IL-1β, and IL-1Ra bind three forms of the IL-1 receptor, and 80 kD type I IL-1 receptor (IL-1R1; Sims, J. E., et al., Science 241:585-589 (1988)), a 68 kD type II IL-1 receptor (IL-1RII; McMahan, C. J., et al., EMBO J. 10:2821-2832 (1991)), and a soluble form of the type II IL-1R (sIL-1RII; Colotta, F., et al., Science 261:472-475 (1993)).

The interactions between the IL-1 ligands and receptors play an essential role in the stimulation and regulation of the IL-1-mediated host response to injury and infection. Cells expressing IL-1RI and treated with IL-1α or IL-1β respond in several specific ways, including stimulating nuclear localization of the the rel-related transcription factor NF-κB (for review, see Thanos, D. & Maniatis, T., Cell 80:529-532 (1996)), activation of protein kinases of the mitogen-activated protein kinase superfamily that phosphorylate residue 669 (Thr-669) of the epidermal growth factor receptor (EGFR; Guy, et al., J. Biol. Chem. 267:1846-1852 (1992); Bird, T. A., et al., J. Biol. Chem. 268:22861-22870 (1991); Bird, T. A., et al., J. Biol. Chem. 269:31836-31844 (1994)), and stimulation of transcription of the IL-8 gene (Mukaida, N., et al., J. Biol. Chem. 265:21128-21133 (1990)).

IL-1RI-Like Family.

Many diverse proteins exhibit homology to the cytoplasmic domain of the IL-1RI. This expanding IL-1RI-like family includes mammalian proteins, Drosophila proteins, and a plant (tobacco) protein (Gay, N. J., & Keith, F. J., Nature 351:355-356 (1991); Hashimoto, C., et al., Cell 52:269-279 (1988); Schneider, D. S., et al., Genes & Dev. 120:885-899 (1994); Mitchan, J. L., et al., J. Biol. Chem. 271:5777-5782 (1996)).

The mammalian IL-1RI-like receptor family members include a murine protein MyoD88 (Lord, K. A., et al., Oncogene 5:1095-1097 (1990)) and a human gene rsc786 (Nomura, N., et al., DNA Res. 1:27-35 (1994)). Another murine receptor family member, T1I/ST2, was previously characerized as a novel primary response gene expressed in Balb/c-3T3 cells (Klememz, R., et al., Proc. Natl. Acad. Sci., USA 86:5708-5712 (1989); Tominaga, S. FEBS Lett. 258:301-304 (1989); Tominaga, S., et al., FEBS Lett. 318:83-87 (1993)). The transmembrane protein mulL-IR AcP (Greenfeder, S. A., et al., J. Biol. Chem. 270:13757-13765 (1995)) has homology to both the type I and type II IL-1R. IL-1R AcP has recently been shown to increase the affinity of IL-1RI for IL-1β and may be involved in mediating the IL-1 response.

T1 Receptors.

T1ST2 receptors (hereinafter, “T1 receptors”), as members of the IL-1 receptor family, (Bergers, G., et al., EMBO J. 13:1176 (1994)) have various homologs in different species. In the rat, the T1 receptor homolog is termed Fit-1. Fit-1 is an estrogen-inducible, c-fos-dependent transmembrane protein that shares 26% and 29% amino acid homology to the mouse IL-1RI and IL-1RII receptors, respectively. In mouse and human, Fit-1 is designated ST2 and T1, respectively. The organization of the two IL-1 receptors and the Fit-i/ST2/T1 genes indicates that they are derived from a common ancestor (Sims, J. E., et al., Cytokine 7:483 (1995)). Fit-1 exists in both membrane-bound and soluble forms. The membrane-bound Fit-1 is designated Fit-1M and contains a cytosolic domain similar to that of the IL-1RI. Soluble Fit-1, designated Fit-1S, is secreted and composed only of the extracellular domain of Fit-1M.

In many ways, these two forms of the Fit-1 protein are similar to those of the membrane-bound and soluble IL-1RI. The IL-1sRI is derived from proteolytic cleavage of the cell-bound form (Sims, J. E., et al., Cytokine 7:483 (1995)). Although Fit-1S is identical to the cytosolic portion of Fit-1M, the two isoforms a generated in a manner unlike that of the IL-1RI system. The Fit-1 gene is under the transcriptional direction of two promoters resulting in the differential expression of two Fit-1 isoforms Fit-1M and Fit-1S. Two mRNA transcripts result from alternative RNA splicing of the 3′ end of the Fit-10RF. This is dependent upon alternate promoter usage in the generation of the transcript. Although IL-1b binds weakly to Fit-1 and does not transduce a signal (Reikerstorger, A., et al, J. Biol. Chem. 270:17645 (1995)), a chimeric receptor consisting of the extracellular domain of murine IL-1RI fused to cytosolic domain of Fit-1 can transduce an IL-1 signal (Reikerstorger, A., et al, J. Biol. Chem. 270:17645 (1995)). The cytosolic portion of Fit-I align with GTPase-like sequences of the IL-1RI (Hopp, T. P. Protein Sci. 4:1851 (1995); see below).

IL-1 Production in Various Disease States.

Increased IL-1 production has been reported in patients with various viral, bacterial, fungal, and parasitic infections; intravascular coagulation; high-dose IL-2 therapy; solid tumors; leukemias; Alzheimer's Disease; HIV-1 infection; autoimmune disorders; trauma (including surgery); hemodialysis; ischemic diseases (myocardial infarction); noninfectious hepatitis; asthma; UV radiation; closed head injury; pancreatitis; periodontitis; graft vs. host disease; transplant rejection; and, in healthy subjects, following strenuous exercise. There is an association of increased IL-1β production in patients with Alzheimer's Disease and a possible role for IL-1 in the release of the amyloid precursor protein (Vasilakos, J. P., et al., FEBS Lett. 354:289 (1994)). However, in most conditions, IL-1 is not the only cytokine exhibiting increased production and hence the specificity of the IL-1 findings as related to the pathogenesis of any particular disease is lacking. In various disease states, IL-1β, but not IL-1α, is detected in the circulation.

IL-1 in Therapy.

Although IL-1 has been found to exhibit many important biological activities, it has also been found to be toxic at doses that are close to therapeutic levels (Dinarello, C. A., Blood 87:2095-2147 (1996)). In general, the acute toxicities of either isoform of IL-1 were greater after intravenous injection, when compared with subcutaneous injection. Subcutaneous injection was associated with significant local pale, erythema, and swelling (Kitamura, T. & Takaku, F., Exp. Med. 7:170 (1989); Laughlin, M. J. Ann. Hematol. 67:267 (1993)). Patients receiving intravenous IL-1 at doses of 100 ng/kg or greater experienced significant hypotension. Conversely, subcutaneous injection of IL-1β, within the range of 4-32 ng/kg, resulted in only a single episode of hypotension (at the highest dose; Laughlin, M. J. Ann. Hematol. 67:267 (1993)).

Contrary to IL-1-associated myelostimulation in patients with normal marrow reserves, patients with aplastic anemia treated with five daily doses of IL-1α (30-100 ng/kg) had no increases in peripheral blood counts or bone marrow cellularity (Walsh, C. E., et al., Brit. J. Haematol. 80:106 (1992)). Finally, IL-1 has been administered to patients undergoing various chemotherapeutic regimes to reduce the nadir of neutropenia and thrombocytopenia.

Daily treatment with 40 ng/kg of IL-1α from days 0-13 of autologous bone marrow or stem cells resulted in an earlier recovery of neutropenia (median 12 days; P<0.001; Weisdorf, D., et al., Blood 84:2044 (1994)). After 14 days of treatment, the bone marrow was significantly enriched with committed myeloid progenitor cells. Similar results were reported in patients with AML receiving 50 ng/kg/d of IL-1 for 5 days starting at the time of transplantation with purged or nonpurged bone marrow (Nemunaitis, J., et al, Blood 83:3473 (1994)). Injecting humans with low doses of either IL-1α or IL-1β confirms the impressive pyrogenic and hypotension-inducing properties of the molecules.

Amelioration of Disease Using Soluble IL-1 Receptors.

Administration of murine IL-1sRI to mice has increased the survival of heterotopic allografts and reduced the hyper;lastic lymph node response to allogeneic cells (Fanslow, W. C., et al., Science 248:739 (1990)). In a rat model of antigen-induced arthritis, local instillation of the murine IL-1sRI reduced joint swelling and tissue destruction (Dower, S. K., et al., Ther. Immunol. 1:113 (1994)). These data suggest that the amount of IL-1sRI administered in the normal, contralateral joint was acting immuneically. In a model of experimental autoimmune encephalitis, the IL-1 sRI reduced the severity of the disease (Jacobs, C. A., et al., J. Immunol. 146:2983 (1991)).

Thus, there is a need for polypeptides that function as multifunctional components in the stimulation and regulation of the IL-1-mediated host response to injury and infection. An number of clinical disorders may result, in part, from the alteration of such regulation. Such disorders include various viral, bacterial, fungal, and parasitic infections; intravascular coagulation; those resulting from high-dose IL-2 therapy; solid tumors; leukemias; Alzheimer's Disease; HIV and other retroviral infection; autoimmune disorders; trauma (including surgery); hemodialysis; ischemic diseases (myocardial infarction); noninfectious hepatitis; asthma; UV radiation; closed head injury; pancreatitis; periodontitis; graft vs. host disease; and transplant rejection. As a result, there is a clear need for identification and characterization of human polypeptides which can play a role in detecting, preventing, ameliorating or correcting such disorders.

### SUMMARY OF THE INVENTION

The present invention provides isolated nucleic acid molecules comprising two highly related polynucleotides, splice variants of each other, each of which encodes a human T1-recpetor (T1-R)-like polypeptide having the complete amino acid sequence shown in SEQ ID NO:2 or SEQ ID NO:4 or the complete amino acid sequences encoded by the cDNA clones deposited as plasmid DNA as ATCC Deposit Number 97859 on Feb. 7, 1997 or ATCC 97858, also on Feb. 7, 1997. The ATCC is located at American Type Culture collection (ATCC), 12301 Park Lawn Drive, Rockville, Md. 20852, USA. The nucleotide sequence determined by sequencing the deposited T1-R-like Ligand III clone, which is shown in FIG. 1 (SEQ ID NO: 1), contains an open reading frame encoding a complete polypeptide of about 163 amino acid residues, including an N-terminal methionine at nucleotide positions 31-33, a leader sequence of about 24 amino acids, and a predicted molecular weight of about 18 kD. The nucleotide sequence determined by sequencing the deposited T1-R-like Ligand III clone, which is shown in FIG. 2 (SEQ ID NO:3), contains an open reading frame encoding a complete polypeptide of about 215 amino acid residues, including an N-terminal methionine at nucleotide positions 31-33, a leader sequence of about 24 amino acids, and a predicted molecular weight of about 24 kD.

Nucleic acid molecules of the invention include those encoding the complete amino acid sequence excepting the N-terminal methionine shown in SEQ ID NO:2 or SEQ ID NO:4, or the complete amino acid sequence excepting the N-terminal methionine encoded by the cDNA clone in ATCC Deposit Number 97859 or in ATCC Deposit Number ATCC 97858, which molecules also can encode additional amino acids fused to the N-terminus of the T1-R-like ligand III amino acid sequence or the T1-R-like ligand III splice variant amino acid sequence.

The encoded polypeptide has a predicted leader sequence of 24 amino acids underlined in FIG. 1 or FIG. 4; and the amino acid sequence of the predicted mature T1-R ligand III protein is also shown in FIG. 1, as amino acid residues 25-163 and as residues 1-139 in SEQ ID NO:2 and in FIG. 4, as amino acid residues 25-215 and as residues 1-191 in SEQ ID NO:4.

Thus, one aspect of the invention provides an isolated nucleic acid molecule comprising a polynucleotide comprising a nucleotide sequence selected from the group consisting of: (a) a nucleotide sequence encoding the T1-R ligand III polypeptide having the complete amino acid sequence in SEQ ID NO:2 or in SEQ ID NO:4 (i.e., positions -24 to 139 of SEQ ID NO:2 or positions -24 to 191 in SEQ ID NO:4); (b) a nucleotide sequence encoding the T1-R ligand III polypeptide having the complete amino acid sequence in SEQ ID NO:2 or in SEQ ID NO:4 excepting the N-terminal methionine (i.e., positions −23 to 139 of SEQ ID NO:2 or positions −23 to 191 in SEQ ID NO:4); (c) a nucleotide sequence encoding the predicted mature T1-R ligand III polypeptide having the amino acid sequence at positions 1-139 in SEQ ID NO:2 or at positions 1-191 in SEQ ID NO:4; (d) a nucleotide sequence encoding the T1-R ligand III polypeptide having the complete amino acid sequence excepting the N-terminal methionine encoded by the cDNA clone contained in ATCC Deposit No. 97859 or in ATCC Deposit No.ATCC 97858; (e) a nucleotide sequence encoding the mature T1-R ligand III polypeptide having the amino acid sequence encoded by the cDNA clone contained in ATCC Deposit No. 97859 or in ATCC Deposit No. ATCC 97858; and (f) a nucleotide sequence complementary to any of the nucleotide sequences in (a), (b), (c), (d) or (e) above.

An additional embodiment of this aspect of the invention relates to a peptide or polypeptide which comprises the amino acid sequence of an epitope-bearing portion of a T1-R ligand III polypeptide having an amino acid sequence described in (a), (b), (c), (d) or (e), above. Peptides or polypeptides having the amino acid sequence of an epitope-bearing portion of a T1-R ligand III polypeptide of the invention include portions of such polypeptides with at least six or seven, preferably at least nine, and more preferably at least about 30 amino acids to about 50 amino acids, although epitope-bearing polypeptides of any length up to and including the entire amino acid sequence of a polypeptide of the invention described above also are included in the invention.

In another embodiment, the invention provides an isolated antibody that binds specifically to a T1-R ligand III polypeptide having an amino acid sequence described in (a), (b), (c), (d) or (e) above. The invention further provides methods for isolating antibodies that bind specifically to a T1-R ligand III polypeptide having an amino acid sequence as described herein. Such antibodies are useful diagnostically or therapeutically as described below.

The invention also provides for pharmaceutical compositions comprising T1-R ligand III polypeptides, particularly human T1-R ligand III polypeptides, which may be employed, for instance, to treat leukemia, atheroscerosis, autoimmune disease, inflammation, metabolic dysfunction or other immune-mediated diseases. Methods of treating individuals in need of T1-R ligand III polypeptides are also provided.

The invention further provides compositions comprising a T1-R ligand III polynucleotide or an T1-R ligand III polypeptide for administration to cells in vitro, to cells ex vivo and to cells in vivo, or to a multicellular organism. In certain particularly preferred embodiments of this aspect of the invention, the compositions comprise a T1-R ligand III polynucleotide for expression of a T1-R ligand III polypeptide in a host organism for treatment of disease. Particularly preferred in this regard is expression in a human patient for treatment of a dysfunction associated with aberrant endogenous activity of a T1-R ligand III.

The present invention also provides a screening method for identifying compounds capable of enhancing or inhibiting a biological activity of the T1-R ligand III polypeptide, which involves contacting a receptor which is enhanced by the T1-R ligand III polypeptide with the candidate compound in the presence of a T1-R ligand III polypeptide, assaying signal transduction activity of the receptor in the presence of the candidate compound and of T1-R ligand III polypeptide, and comparing the receptor activity to a standard level of activity, the standard being assayed when contact is made between the receptor and in the presence of the T1-R ligand III polypeptide and the absence of the candidate compound In this assay, an increase in receptor activity over the standard indicates that the candidate compound is an agonist of T1-R ligand III activity and a decrease in receptor activity compared to the standard indicates that the compound is an antagonist of T1-R ligand III activity.

In another aspect, a screening assay for agonists and antagonists is provided which involves determining the effect a candidate compound has on T1-R ligand III binding to a T1-receptor or homolog thereof. In particular, the method involves contacting the T1-receptor or homolog thereof with a T1-R ligand III polypeptide and a candidate compound and determining whether T1-R ligand III polypeptide binding to the T1-receptor or homolog thereof is increased or decreased due to the presence of the candidate compound. In this assay, an increase in binding of T1-R ligand III over the standard binding indicates that the candidate compound is an agonist of T1-R ligand III binding activity and a decrease in T1-R ligand III binding compared to the standard indicates that the compound is an antagonist of T1-R ligand III binding activity.

It has been discovered that T1-R ligand III is expressed not only in human eight week old embryo or in human endometrial tumor but also in human adult spleen, TNF-α- and IFN-induced epithelial cells, and keratinocytes. Therefore, nucleic acids of the invention are useful as hybridization probes for differential identification of the tissue(s) or cell type(s) present in a biological sample. Similarly, polypeptides and antibodies directed to those polypeptides are useful to provide immunological probes for differential identification of the tissue(s) or cell type(s). In addition, for a number of disorders of the above tissues or cell s, particularly of the immune system, significantly higher or lower levels of T1-R ligand III gene expression may be detected in certain tissues (e.g., cancerous and wounded tissues) or bodily fluids (e.g., serum, plasma, urine, synovial fluid or spinal fluid) taken from an individual having such a disorder, relative to a “standard” T1-R ligand III gene expression level, i.e., the T1-R ligand III expression level in healthy tissue from an individual not having the immune system disorder. Thus, the invention provides a diagnostic method useful during diagnosis of such a disorder, which involves: (a) assaying T1-R ligand III gene expression level in cells or body fluid of an individual; (b) comparing the T1-R ligand III gene expression level with a standard T1-R ligand III gene expression level, whereby an increase or decrease in the assayed T1-R ligand III gene expression level compared to the standard expression level is indicative of disorder in the immune system.

An additional aspect of the invention is related to a method for treating an individual in need of an increased level of T1-R ligand III activity in the body comprising administering to such an individual a composition comprising a therapeutically effective amount of an isolated T1-R ligand III polypeptide of the invention or an agonist thereof.

A still further aspect of the invention is related to a method for treating an individual in need of a decreased level of T1-R ligand III activity in the body comprising, administering to such an individual a composition comprising a therapeutically effective amount of an T1-R ligand III antagonist. Preferred antagonists for use in the present invention are T1-R ligand III-specific antibodies.

### BRIEF DESCRIPTION OF THE FIGURES

FIG. 1 shows the nucleotide sequence (SEQ ID NO:1) and deduced amino acid sequence (SEQ ID NO:2) of T1-R ligand III.

The predicted leader sequence of about 24 amino acids is underlined. Note that the methionine residue at the beginning of the leader sequence in FIG. 1 is shown in position number (positive) 1, whereas the leader positions in the corresponding sequence of SEQ ID NO:2 are designated with negative position numbers. Thus, the leader sequence positions 1 to 24 in FIG. 1 correspond to positions −24 to −1 in SEQ ID NO:2.

FIG. 2 shows the nucleotide sequence (SEQ ID NO:3) and deduced amino acid sequence (SEQ ID NO:4) of the T1-R ligand III splice variant.

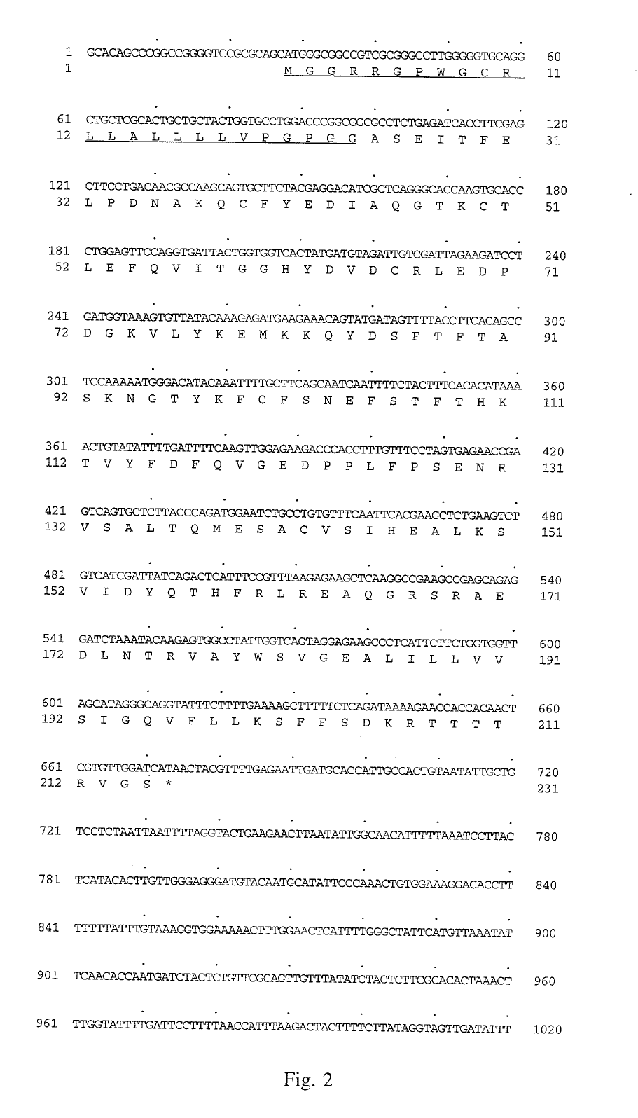

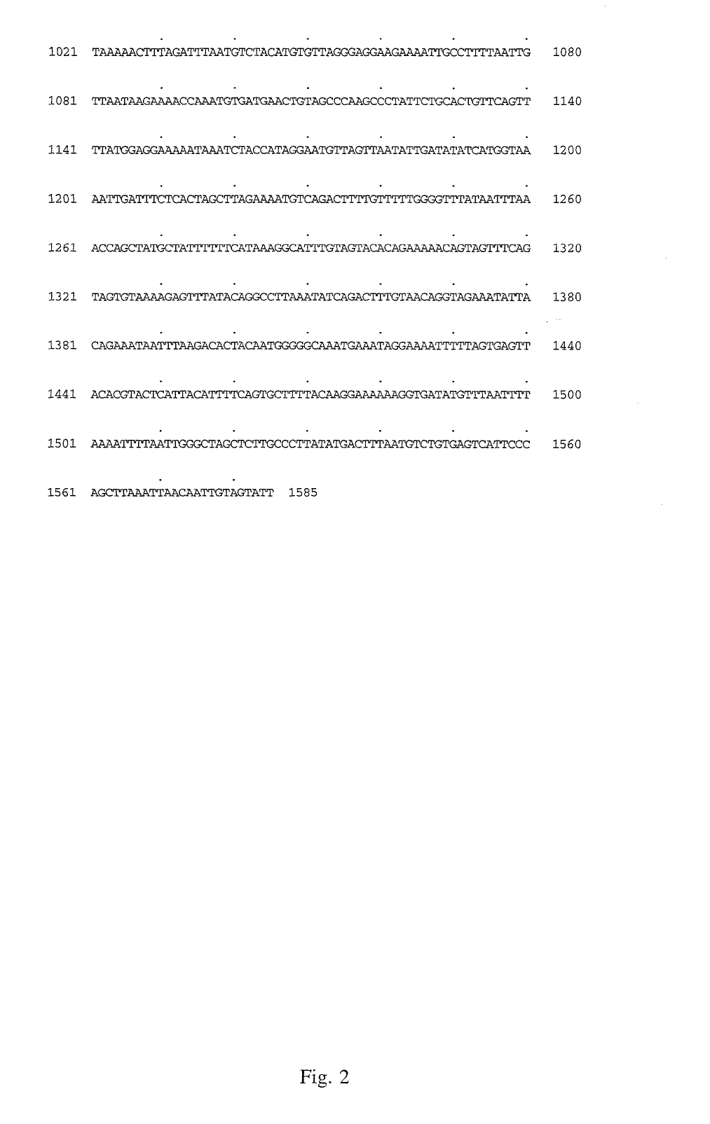

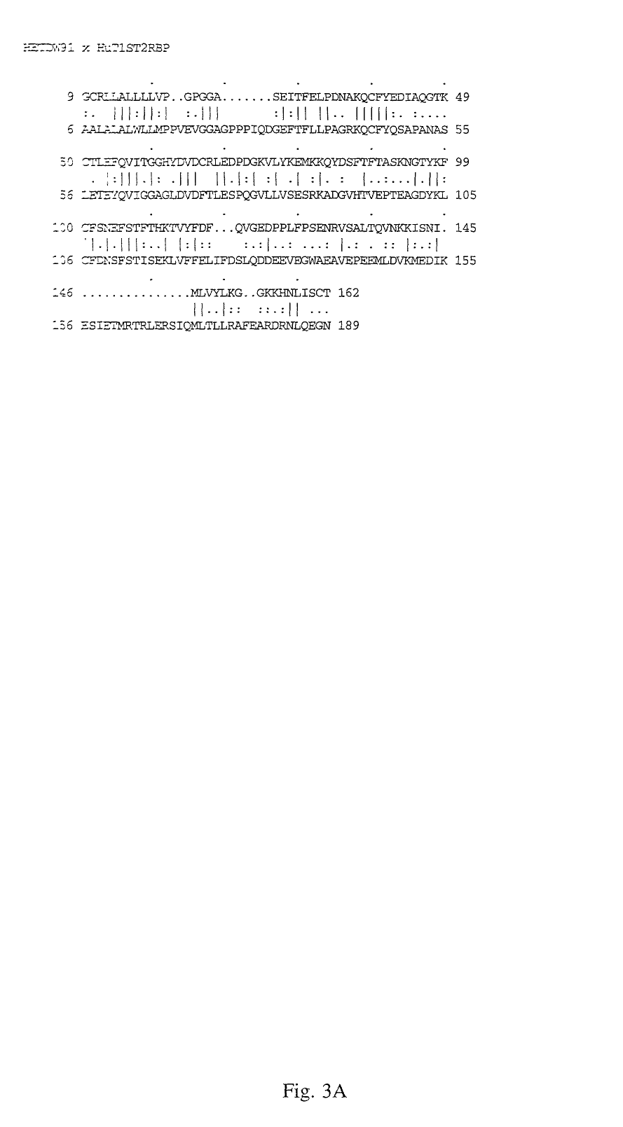

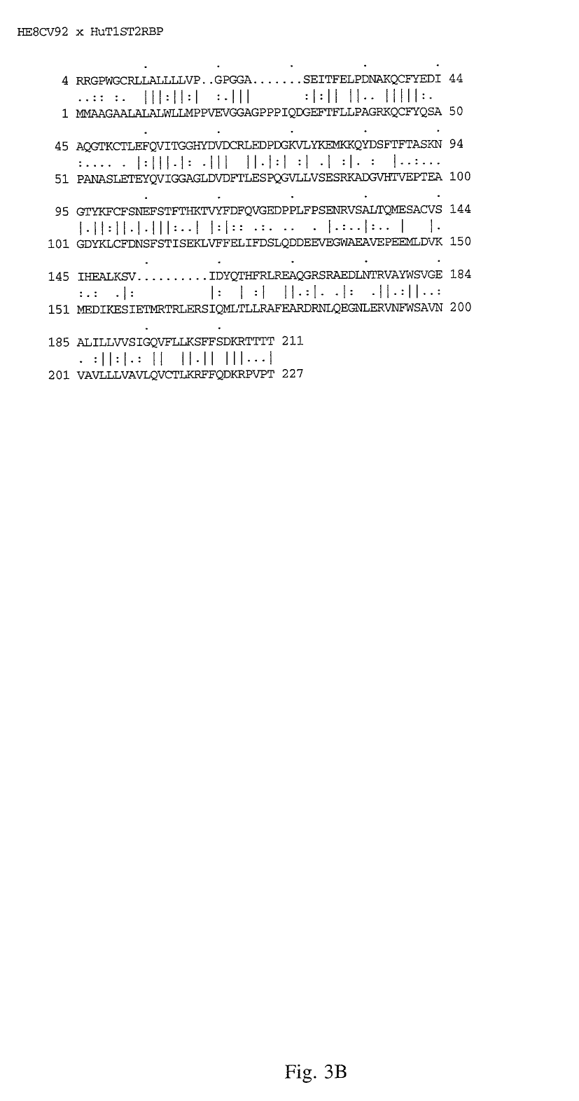

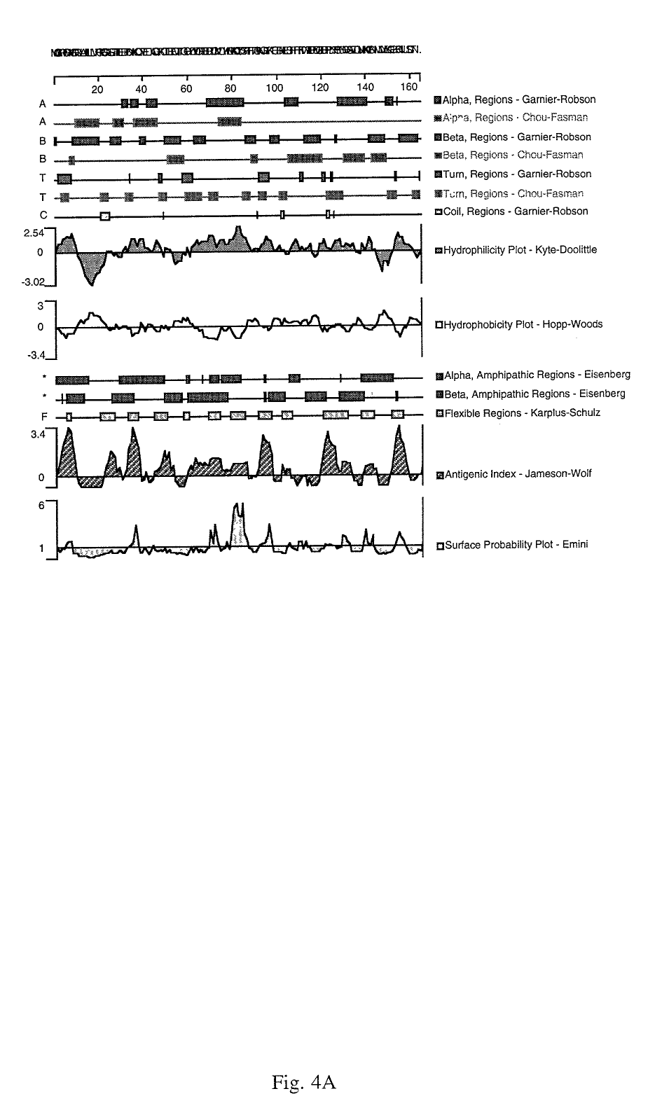

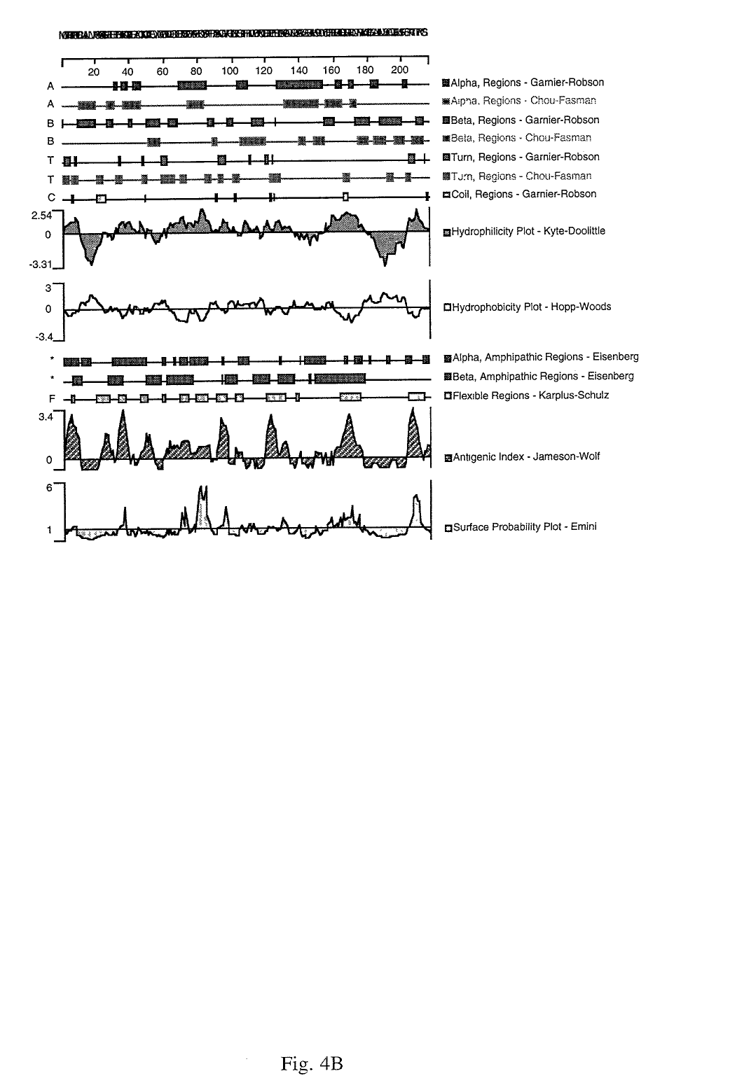

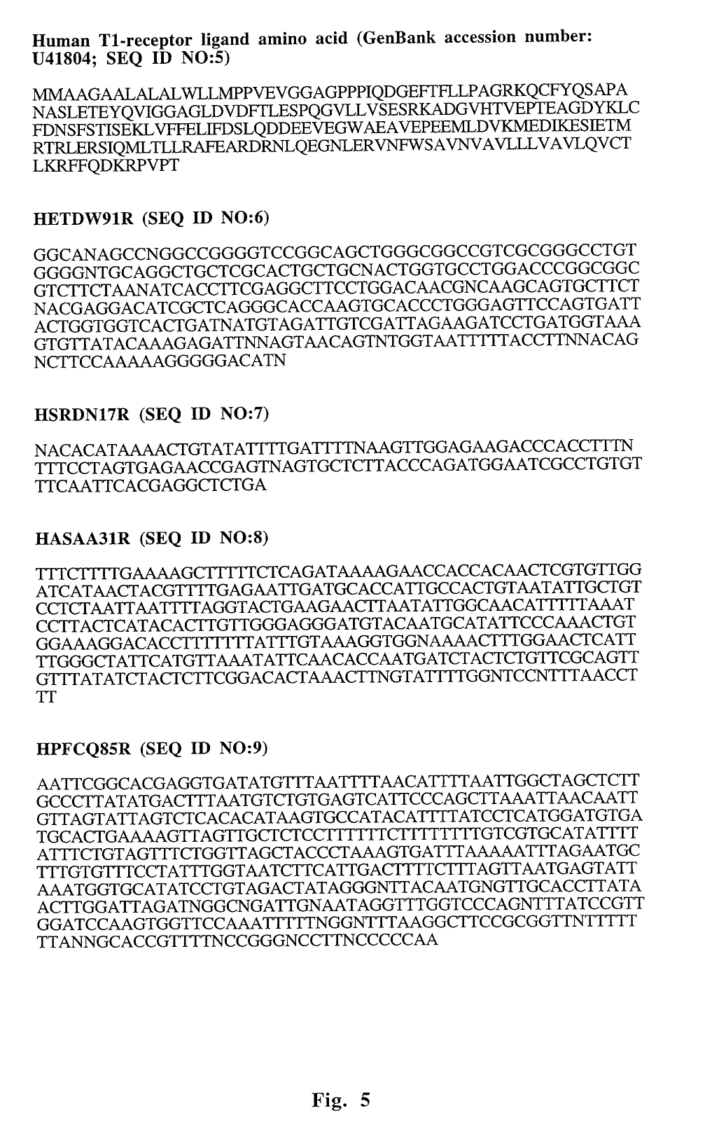

The predicted leader sequence of about 24 amino acids is underlined. Note that the methionine residue at the beginning of the leader sequence in FIG. 2 is shown in position number (positive) 1, whereas the leader positions in the corresponding sequence of SEQ ID NO:4 are designated with negative position numbers. Thus, the leader sequence positions 1 to 24 in FIG. 2 correspond to positions −24 to −1 in SEQ ID NO:4. The region of T1-R ligand III present in the splice variant listed in SEQ ID NO:4 which is not present in the splice variant listed in SEQ ID NO:2, positions 180-203 of SEQ ID NO:4, is also underlined in FIG. 2.

FIG. 3A shows the regions of identity between the amino acid sequences of the T1-R ligand III protein (SEQ ID NO:2) and translation product of the human mRNA for T1-receptor ligand (SEQ ID NO:5), determined by the computer program BestFit (Wisconsin Sequence Analysis Package, Version 8 for Unix, Genetics Computer Group, University Research Park, 575 Science Drive, Madison, WI 53711) using the default parameters. FIG. 3B shows the regions of identity between the amino acid sequences of the T1-R ligand III protein (SEQ ID NO:4) and translation product of the human mRNA for T1-receptor ligand (SEQ ID NO:5), determined by BestFit.

FIGS. 4A and 4B show a analyses of the T1-R ligand III amino acid sequences listed in SEQ ID NO:2 and SEQ ID NO:4, respectively. Alpha, beta, turn and coil regions; hydrophilicity and hydrophobicity; amphipathic regions; flexible regions; antigenic index and surface probability are shown. In the “Antigenic Index—Jameson-Wolf” graph, the positive peaks indicate locations of the highly antigenic regions of the T1-R ligand III protein, i.e., regions from which epitope-bearing peptides of the invention can be obtained.

FIG. 5 shows the amino acid sequence of the human T1-R ligand (GenBank U41804) and the nucleotide sequence of expressed sequence tag (EST) cDNA clones which share sequence identity with T1-R Ligand III (SEQ ID NO:2 and SEQ ID NO:4).

### DETAILED DESCRIPTION

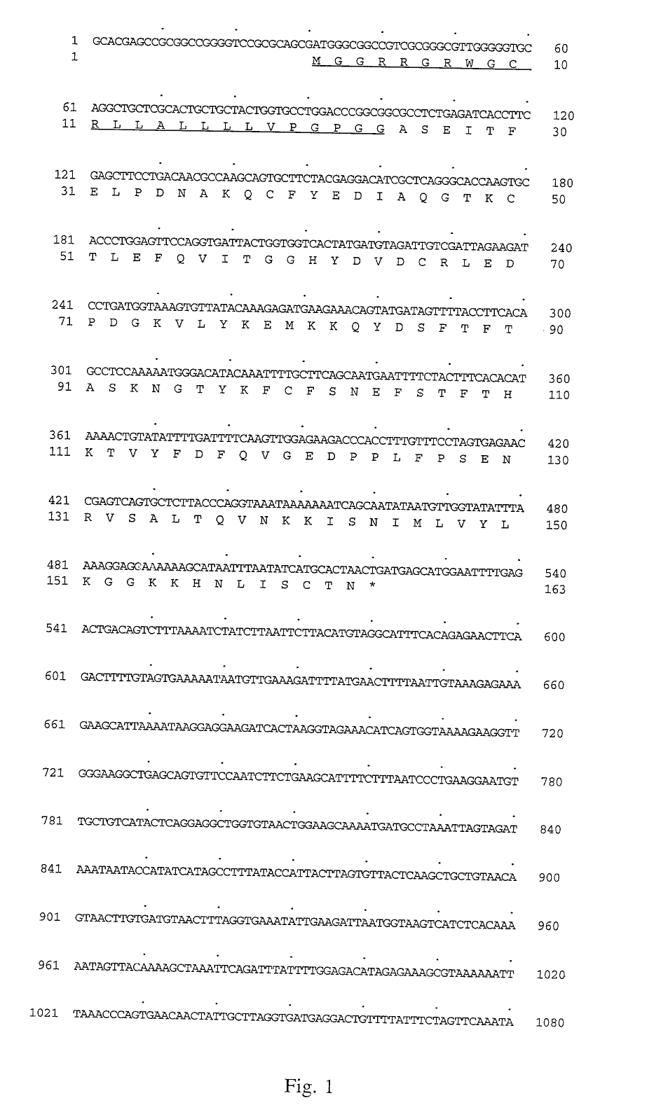

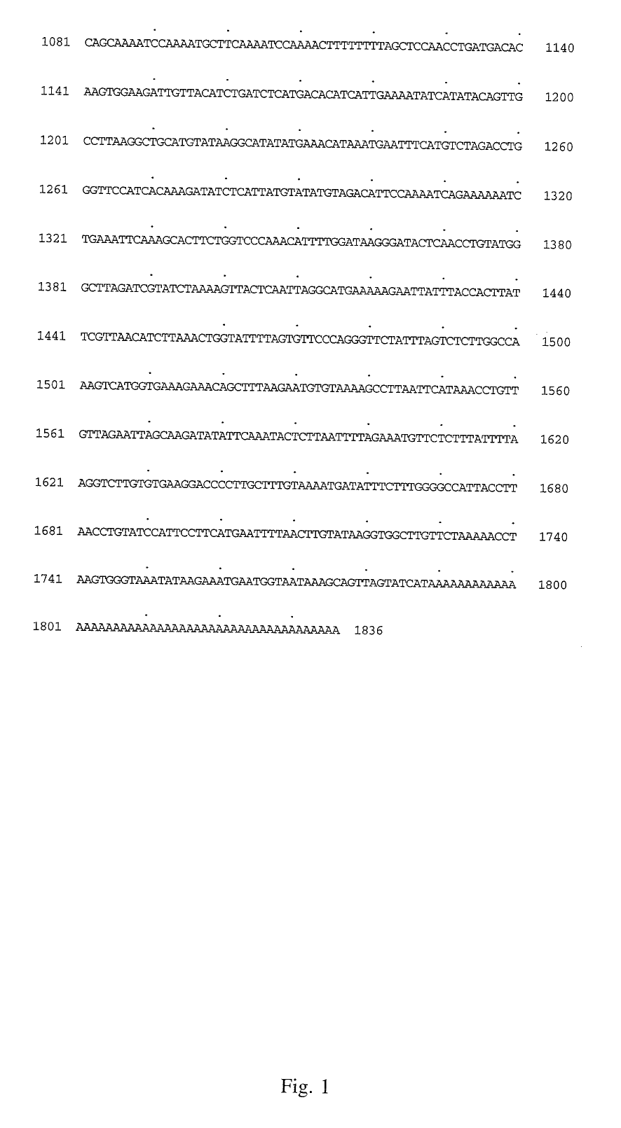

The present invention provides isolated nucleic acid molecules comprising a polynucleotide encoding a T1-R ligand III polypeptide having the amino acid sequence shown in SEQ ID NO:2 or in SEQ ID NO:4, which was determined by sequencing a cloned cDNA. The nucleotide sequence shown in FIG. 1 (SEQ ID NO:1) or FIG. 2 (SEQ ID NO:3) was obtained by sequencing the HETDW91 clone (1631434) or the HE8CV92 clone 1631438), which were deposited on Feb. 7, 1997 at the American Type Culture Collection, 12301 Park Lawn Drive, Rockville, Md. 20852, and given accession number ATCC 97859 or accession number ATCC 97858. The deposited clones are contained in the pBluescript SK(−) plasmid (Stratagene, La Jolla, Calif.).

The T1-R ligand III protein of the present invention shares sequence homology with the translation product of the human mRNA for T1-R ligand (SEQ ID NO:3; FIG. 3A or FIG. 3B). T1-R ligand shares significant sequence homology with the cytoplasmic domain of the IL-1RI. The mammalian IL-1RI-like receptor family members include a murine protein MyoD88 (Lord, K. A., et al., Oncogene 5:1095-1097 (1990)) and a human gene rsc786 (Nomura, N., et al., DNA Res. 1:27-35 (1994)). Another murine receptor family member, T1/ST2, was previously characerized as a novel primary response gene expressed in Balb/c-3T3 cells (Klememz, R., et al., Proc. Natl. Acad. Sci., USA 86:5708-5712 (1989); Tominaga, S. FEBS Lett. 258:301-304 (1989); Tominaga, S., et al., FEBS Lett. 318:83-87 (1993)). The transmembrane protein mulL-IR AcP (Greenfeder, S. A., et al., J. Biol. Chem. 270:13757-13765 (1995)) has homology to both the type I and type II IL-1R. IL-IR AcP has recently been shown to increase the affinity of IL-1RI for IL-1β and may be involved in mediating the IL-I response.

### Nucleic Acid Molecules

Unless otherwise indicated, all nucleotide sequences determined by sequencing a DNA molecule herein were determined using an automated DNA sequencer (such as the Model 373 from Applied Biosystems, Inc., Foster City, Calif.), and all amino acid sequences of polypeptides encoded by DNA molecules determined herein were predicted by translation of a DNA sequence determined as above. Therefore, as is known in the art for any DNA sequence determined by this automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art. As is also known in the art, a single insertion or deletion in a determined nucleotide sequence compared to the actual sequence will cause a frame shift in translation of the nucleotide sequence such that the predicted amino acid sequence encoded by a determined nucleotide sequence will be completely different from the amino acid sequence actually encoded by the sequenced DNA molecule, beginning at the point of such an insertion or deletion.

By “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U), where each thymidine deoxyribonucleotide (T) in the specified deoxyribonucleotide sequence is replaced by the ribonucleotide uridine (U).

Using the information provided herein, such as the nucleotide sequence in FIG. 1 (SEQ ID NO:1) or FIG. 2 (SEQ ID NO:3), a nucleic acid molecule of the present invention encoding a T1-R ligand III polypeptide may be obtained using standard cloning and screening procedures, such as those for cloning cDNAs using mRNA as starting material. Illustrative of the invention, the nucleic acid molecule described in FIG. 1 (SEQ ID NO:1) or FIG. 2 (SEQ ID NO:3) was discovered in a cDNA library derived from human endometrial tumor or from human eight week old whole embryo.

Additional clones of the same gene were also identified in cDNA libraries constructed from human adult spleen tissue, TNF-α- and IFN-induced epithelial cells, and keratinocytes.

The determined nucleotide sequence of the T1-R ligand III cDNA of FIG. 1 (SEQ ID NO:1) contains an open reading frame encoding a protein of 163 amino acid residues, with an initiation codon at nucleotide positions 31-33 of the nucleotide sequence in FIG. 1 (SEQ ID NO:1), and a deduced molecular weight of about 18 kD. The determined nucleotide sequence of the T1-R ligand III cDNA of FIG. 2 (SEQ ID NO:3) contains an open reading frame encoding a protein of 215 amino acid residues, with an initiation codon at nucleotide positions 31-33 of the nucleotide sequence in FIG. 2 (SEQ ID NO:3), and a deduced molecular weight of about 24 kD. The amino acid sequence of the T1-R ligand III protein shown in SEQ ID NO:2 is about 36% identical to human mRNA for T1-R ligand (SEQ ID NO:5; FIG. 3A; Gayle, M. A., et al., J. Biol. Chem. 271:5784-5789 (1996); GenBank accession number U41804). The amino acid sequence of the T1-R ligand III protein shown in SEQ ID NO:4 is also about 36% identical to human mRNA for T1-R ligand (FIG. 3B).

Leader and Mature Sequences

The amino acid sequence of the complete T1-R ligand III protein includes a leader sequence and a mature protein, as shown in SEQ ID NO:2 or SEQ ID NO:4. More in particular, the present invention provides nucleic acid molecules encoding a mature form of the T1-R ligand III protein. Thus, according to the signal hypothesis, once export of the growing protein chain across the rough endoplasmic reticulum has been initiated, proteins secreted by mammalian cells have a signal or secretory leader sequence which is cleaved from the complete polypeptide to produce a secreted “mature” form of the protein. Most mammalian cells and even insect cells cleave secreted proteins with the same specificity. However, in some cases, cleavage of a secreted protein is not entirely uniform, which results in two or more mature species of the protein. Further, it has long been known that the cleavage specificity of a secreted protein is ultimately determined by the primary structure of the complete protein, that is, it is inherent in the amino acid sequence of the polypeptide. Therefore, the present invention provides a nucleotide sequence encoding the mature T1-R ligand III polypeptide having the amino acid sequence encoded by the cDNA clone contained in the plasmid DNA identified as ATCC Deposit No. 97859 or ATCC Deposit No. 97858. By the “mature T1-R ligand III polypeptide having the amino acid sequence encoded by the cDNA clone in ATCC Deposit No. 97859 or in ATCC Deposit No. 97858” is meant the mature form(s) of the T1-R ligand III protein produced by expression in a mammalian cell (e.g., COS cells, as described below) of the complete open reading frame encoded by the human DNA sequence of the clone contained in the vector in the deposited DNA.

In addition, methods for predicting whether a protein has a secretory leader as well as the cleavage point for that leader sequence are available. For instance, the method of McGeoch (Virus Res. 3:271-286 (1985)) uses the information from a short N-terminal charged region and a subsequent uncharged region of the complete (uncleaved) protein. The method of von Heinje (Nucleic Acids Res. 14:4683-4690 (1986)) uses the information from the residues surrounding the cleavage site, typically residues −13 to +2 where +1 indicates the amino terminus of the mature protein. The accuracy of predicting the cleavage points of known mammalian secretory proteins for each of these methods is in the range of 75-80% (von Heinje, supra). However, the two methods do not always produce the same predicted cleavage point(s) for a given protein.

In the present case, the deduced amino acid sequence of the complete T1-R ligand III polypeptide was analyzed by a computer program PSORT (Nakai, K. & Kanehisa, M., Genomics 14:897-911 (1992)), which is an expert system for predicting the cellular location of a protein based on the amino acid sequence. As part of this computational prediction of localization, the methods of McGeoch and von Heinje are incorporated. The analysis of the T1-R ligand III amino acid sequence by this program indicated a likelihood that the sequence provided in SEQ ID NO:2 contains a cleavable N-terminal signal sequence. The analysis of the T1-R ligand III amino acid sequence by this program also indicated a likelihood that the sequence provided in SEQ ID NO:4 contains a cleavable N-terminal signal sequence.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at positions 31-33 of the nucleotide sequence shown in FIG. 1 (SEQ ID NO:1) or in FIG. 2 (SEQ ID NO:3).

Also included are DNA molecules comprising the coding sequence for the predicted mature T1-R ligand III protein shown at positions 1-139 of SEQ ID NO:2 and at positions 1-191 of SEQ ID NO:4.

In addition, isolated nucleic acid molecules of the invention include DNA molecules which comprise a sequence substantially different from those described above but which, due to the degeneracy of the genetic code, still encode the T1-R ligand III protein. Of course, the genetic code and species-specific codon preferences are well known in the art. Thus, it would be routine for one skilled in the art to generate the degenerate variants described above, for instance, to optimize codon expression for a particular host (e.g., change codons in the human mRNA to those preferred by a bacterial host such as E. coli).

In another aspect, the invention provides isolated nucleic acid molecules encoding the T1-R ligand III polypeptide having an amino acid sequence encoded by the cDNA clone contained in the plasmid deposited as ATCC Deposit No. 97859 on Feb. 7, 1997 or 97858 also on Feb. 7, 1997.

Preferably, this nucleic acid molecule will encode the mature polypeptide encoded by the above-described deposited cDNA clone.

The invention further provides an isolated nucleic acid molecule having the nucleotide sequence shown in FIG. 1 (SEQ ID NO:1) or FIG. 2 (SEQ ID NO:3) or the nucleotide sequence of the T1-R ligand III cDNA contained in the above-described deposited clones, or a nucleic acid molecule having a sequence complementary to one of the above sequences. Such isolated molecules, particularly DNA molecules, are useful as probes for gene mapping, by in situ hybridization with chromosomes, and for detecting expression of the T1-R ligand III gene in human tissue, for instance, by Northern blot analysis.

The present invention is further directed to nucleic acid molecules encoding portions of the nucleotide sequences described herein as well as to fragments of the isolated nucleic acid molecules described herein. In particular, the invention provides a polynucleotide having a nucleotide sequence representing the portion of SEQ ID NO:1 which consists of positions 1-1836 of SEQ ID NO:1 or of positions 1-1585 of SEQ ID NO:3.

In addition, the invention provides a nucleic acid molecule having a nucleotide sequence related to a portion of SEQ ID NO:1 which has been determined from the following related cDNA clones (FIG. 5): HETDW91R (SEQ ID NO: 6) and HSRDN17R (SEQ ID NO:7). The cDNA clones HETDW91R (SEQ ID NO: 6) and HSRDN17R (SEQ ID NO:7) exhibits approximately 97% and 95% sequence identity to nucleotides 130-269 and 355-441 of T1-R ligand III (SEQ ID NO:1), respectively. The invention also provides nucleic acid molecules having nucleotide sequences related to portions of SEQ ID NO:3 which have been determined from the following related cDNA clones (FIG. 5): HETDW91R (SEQ ID NO:6), HSRDN17R (SEQ ID NO:7), HASAA31R (SEQ ID NO:8), and HPFCQ85R (SEQ ID NO:9). The homologies between each of these clones and the determined sequence of the deposited of positions 1-1585 of SEQ ID NO:3 cDNA clone are as follows. The cDNA clones HASAA31R (SEQ ID NO:7), HSRDN17R (SEQ ID NO:8), and HPFCQ85R (SEQ ID NO:9) exhibit approximately 97%, 96%, 96%, and 100% sequence identity to nucleotides 127-266, 352-475, 616-983, and 1513-1579 of T1-R ligand III (SEQ ID NO: 1), respectively.

Further, the invention includes a polynucleotide comprising any portion of at least about 30 nucleotides, preferably at least about 50 nucleotides, of SEQ ID NO:1 from residues 1 to 100, 450 to 615, and 690 to 1836. More preferably, the invention includes a polynucleotide comprising nucleotide residues 610 to 700, 600 to 750, 500 to 1000, 95 to 750, 90 to 1000, 85 to 1500, 80 to 1700, 75 to 1750, and 50 to 1800 of SEQ ID NO:1.

The invention also includes a polynucleotide comprising any portion of at least about 30 nucleotides, preferably at least about 50 nucleotides, of SEQ ID NO:2 from residues 1 to 100, 1000 to 1390, and 1430 to 1513. More preferably, the invention includes a polynucleotide comprising nucleotide residues 1350 to 1450, 1350 to 1500, 1 100 to 1300, 95 to 1350, 75 to 1450, and 50 to 1500 of SEQ ID NO:3.

More generally, by a fragment of an isolated nucleic acid molecule having the nucleotide sequence of the deposited cDNA or the nucleotide sequence shown in FIG. 1 (SEQ ID NO:1) is intended fragments at least about 15 nt, and more preferably at least about 20 nt, still more preferably at least about 30 nt, and even more preferably, at least about 40 nt in length which are useful as diagnostic probes and primers as discussed herein. Of course, larger fragments 50-300 nt in length are also useful according to the present invention as are fragments corresponding to most, if not all, of the nucleotide sequence of the deposited cDNA or as shown in FIG. 1 (SEQ ID NO:1). By a fragment at least 20 nt in length, for example, is intended fragments which include 20 or more contiguous bases from the nucleotide sequence of the deposited cDNA or the nucleotide sequence as shown in FIG. 1 (SEQ ID NO:1). Preferred nucleic acid fragments of the present invention include nucleic acid molecules encoding epitope-bearing portions of the T1-R ligand III polypeptide as identified in FIG. 3A and described in more detail below.

In another aspect, the invention provides an isolated nucleic acid molecule comprising a polynucleotide which hybridizes under stringent hybridization conditions to a portion of the polynucleotide in a nucleic acid molecule of the invention described above, for instance, the cDNA clone contained in ATCC Deposit No. 97859 or in ATCC Deposit No. 97858. By “stringent hybridization conditions” is intended overnight incubation at 42° C. in a solution comprising: 50% formamide, 5× SSC (150 mM NaCl, 15 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1× SSC at about 65° C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 (e.g., 50) nt of the reference polynucleotide. These are useful as diagnostic probes and primers as discussed above and in more detail below.

By a portion of a polynucleotide of “at least 20 nt in length,” for example, is intended 20 or more contiguous nucleotides from the nucleotide sequence of the reference polynucleotide (e.g., the deposited cDNA or the nucleotide sequence as shown in FIG. 1 (SEQ ID NO: 1) or the nucleotide sequence as shown in FIG. 2 (SEQ ID NO:3)). Of course, a polynucleotide which hybridizes only to a poly A sequence (such as the 3′ terminal poly(A) tract of the T1-R ligand III cDNA shown in FIG. 1 (SEQ ID NO:1)), or to a complementary stretch of T (or U) residues, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention which encode a T1-R ligand III polypeptide may include, but are not limited to those encoding the amino acid sequence of the mature polypeptide, by itself; and the coding sequence for the mature polypeptide and additional sequences, such as those encoding the about 24 amino acid leader or secretory sequence, such as a pre-, or pro- or prepro-protein sequence; the coding sequence of the mature polypeptide, with or without the aforementioned additional coding sequences.

Also encoded by nucleic acids of the invention are the above protein sequences together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; an additional coding sequence which codes for additional amino acids, such as those which provide additional functionalities.

Thus, the sequence encoding the polypeptide may be fused to a marker sequence, such as a sequence encoding a peptide which facilitates purification of the fused polypeptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described by Gentz and coworkers (Proc. Natl. Acad. Sci. USA 86:821-824 (1989)), for instance, hexa-histidine provides for convenient purification of the fusion protein. The “HA” tag is another peptide useful for purification which corresponds to an epitope derived from the influenza hemagglutinin protein, which has been described by Wilson and colleagues (Cell 37: 767 (1984)). As discussed below, other such fusion proteins include the T1-R ligand III fused to Fc at the N- or C-terminus. Variant and Mutant Polynucleotides The present invention further relates to variants of the nucleic acid molecules of the present invention, which encode portions, analogs or derivatives of the T1-R ligand III protein. Variants may occur naturally, such as a natural allelic variant. By an “allelic variant” is intended one of several alternate forms of a gene occupying a given locus on a chromosome of an organism. (Genes II, Lewin, B., ed., John Wiley & Sons, New York (1985)). Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the T1-R ligand III protein or portions thereof. Also especially preferred in this regard are conservative substitutions.

Most highly preferred are nucleic acid molecules encoding the mature protein having the amino acid sequence shown in SEQ ID NO:2 or in SEQ ID NO:4 or the mature T1-R ligand III amino acid sequence encoded by the deposited cDNA clones.

Further embodiments of the invention include isolated nucleic acid molecules that comprise a polynucleotide having a nucleotide sequence at least 90% identical, and more preferably at least 95%, 96%, 97%, 98% or 99% identical, to any of the nucleotide sequences in (a), (b), (c), (d), (e) or (f), above, or a polynucleotide which hybridizes under stringent hybridization conditions to a polynucleotide in (a), (b), (c), (d), (e) or (f), above. This polynucleotide which hybridizes does not hybridize under stringent hybridization conditions to a polynucleotide having a nucleotide sequence consisting of only A residues or of only T residues. An additional nucleic acid embodiment of the invention relates to an isolated nucleic acid molecule comprising a polynucleotide which encodes the amino acid sequence of an epitope-bearing portion of a T1-R ligand III polypeptide having an amino acid sequence in (a), (b), (c), (d) or (e), above.

The present invention also relates to recombinant vectors, which include the isolated nucleic acid molecules of the present invention, and to host cells containing the recombinant vectors, as well as to methods of making such vectors and host cells and for using them for production of T1-R ligand III polypeptides or peptides by recombinant techniques.

By a polynucleotide having a nucleotide sequence at least, for example, 95% “identical” to a reference nucleotide sequence encoding a T1-R ligand III polypeptide is intended that the nucleotide sequence of the polynucleotide is identical to the reference sequence except that the polynucleotide sequence may include up to five point mutations per each 100 nucleotides of the reference nucleotide sequence encoding the T1-R ligand III polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIG. 1 or FIG. 2 or to the nucleotides sequence of the deposited cDNA clone can be determined conventionally using known computer programs such as the Bestfit program (Wisconsin Sequence Analysis Package, Version 8 for Unix, Genetics Computer Group, University Research Park, 575 Science Drive, Madison, WI 53711). Bestfit uses the local homology algorithm of Smith and Waterman, (Advances in Applied Mathematics 2:482-489 (1981)), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 90%, 95%, 96%, 97%, 98% or 99% identical to the nucleic acid sequence shown in FIG. 1 (SEQ ID NO:1) or to the nucleic acid sequence shown in FIG. 2 (SEQ ID NO:3) or to the nucleic acid sequence of the deposited cDNA, irrespective of whether they encode a polypeptide having T1-R ligand III activity. This is because even where a particular nucleic acid molecule does not encode a polypeptide having T1-R ligand III activity, one of skill in the art would still know how to use the nucleic acid molecule, for instance, as a hybridization probe or a polymerase chain reaction (PCR) primer. Uses of the nucleic acid molecules of the present invention that do not encode a polypeptide having T1-R ligand III activity include, inter alia, (1) isolating the T1-R ligand III gene or allelic variants thereof in a cDNA library; (2) in situ hybridization (e.g., “FISH”) to metaphase chromosomal spreads to provide precise chromosomal location of the T1-R ligand III gene, as described in Verma and coworkers (Human Chromosomes: A Manual of Basic Techniques, Pergamon Press, New York (1988)); and Northern Blot analysis for detecting T1-R ligand III mRNA expression in specific tissues.

Preferred, however, are nucleic acid molecules having sequences at least 90%, 95%, 96%, 97%, 98% or 99% identical to the nucleic acid sequence shown in FIG. 1 (SEQ ID NO:1) or to the nucleic acid sequence shown in FIG. 2 (SEQ ID NO:3) or to the nucleic acid sequence of the deposited cDNA which do, in fact, encode a polypeptide having T1-R ligand III protein activity. By “a polypeptide having T1-R ligand III activity” is intended polypeptides exhibiting activity similar, but not necessarily identical, to an activity of the mature T1-R ligand III protein of the invention, as measured in a particular biological assay. For example, the T1-R ligand III protein of the present invention can be measured in a particular biological assay. T1-R-ligand III activity can be measured using known receptor binding assays (Mitcham, J. L., et al., J. Biol. Chem. 271:5777-5783 (1996); Gayle, M. A., et al., J. Biol. Chem. 271:5784-5789 (1996)). These assays include an IL-8 promoter activation assay and a peptide kinase assay.

To perform these assays, it is first necessary to transfect mammalian cells with an expression vector containing the cDNA for a suitable receptor. For example, an expression vector containing the cDNA for the previously reported T1/ST2 receptor can be used. This cDNA can be obtained as described (Klemenz, R., et al., Proc. Natl. Acad. Sci. USA 86:5708-5712 (1989); Tominaga, S. FEBS Lett. 258:301-304 (1989); Bergers, G., et al., EMBO J. 13:1176-1188)). Alternatively, T1ST2 cDNA can be amplified using the polymerase chain reaction. A commercially available cDNA library, prepared from mRNA from a suitable tissue or cell type (such as NIH-3T3 cells (Klemenz, R., et al., Proc. Natl. Acad. Sci. USA 86:5708-5712 (1989))) can be used as a template. Using any of several transfection methods, well known to those of ordinary skill in the art, a suitable cell line (e. g. COS7 cells) can be transfected with the T1/ST2 expression plasmid. Expression of the receptor can be verified by radioimmunoassay (see Mitcham, J. L., et al., J. Biol. Chem. 271:5777-5783 (1996)). One to three days post-transfection, confluent transfected COS7 cells are stimulated with 1-10 ng of T1-R ligand III for 15 to 20 hours. Duration of stimulation by T1-R ligand III protein will vary, depending on which assay is used, and can be determined using only routine experimentation.

To perform the biological IL-8 promoter activation assay, nuclear extracts from transfected cells are prepared immediately after stimulation (Ostrowski, J., et al., J. Biol. Chem. 266:12722-12733 (1991)). A double-stranded synthetic oligonucleotide probe containing an NF-KB enhancer element from the immunoglobulin K light chain (e. g. 5′-TGACAGAGGGACTTTCCGAGAGGA-3′) is labled at the 5′ end by phosphorylation with [Y-³²P]-ATP. Nuclear extracts (10 μg) are incubated with radiolabeled probe for 20 minutes at room temperature, and DNA-protein complexes are resolved by electrophoresis in a 0.5× TBE, 10% polyacrylamide gel. Changes in IL-8 promoter activity may be observed indirectly by comparing the DNA-protein complex formation resulting from control and sample preparations.

To perform the in vitro Thr-669 peptide kinase assay, cytoplasmic extracts of transfected cells are prepared immediately after stimulation with T1-R ligand III (Bird, T. A., et al., Cytokine 4:429-440 (1992)). Cellular extract (10 μl) is added to 20 μl of reaction mixture containing 20 mM HEPES buffer (pH 7.4), 15 mM MgCl₂, 15 μM ATP, 75 μCi/ml [γ-³²P]-ATP, and 750 γM substrate peptide (residues 663-673 of the epidermal growth factor receptor). Negative controls are incubated with distilled water in place of the substrate peptide. After incubation at 30 C for 20 minutes, the reactions are terminated by the addition of formic acid. Reactions are cleared by centrifugation, and 30 μl of supernatant are spotted on phosphocellulose disks. After washing three times with 75 mM orthophosphoric acid and drying, the amount of phosphorylated peptideis quantitated by determining Cerenkov counts. Results are expressed as the ratio of Thr-669 kinase activity detected in unstimulated cells when compared to activity detected in stimulated cells.

To perform the IL-8 promoter activation assay, COS7 cells (1×10⁵ cells per well in a 12-well tissue culture plate) are cotransfected with the T1/ST2 receptor expression vector and the pIL8p reporter plasmid (Mitcham, J. L. et al., J. Biol. Chem. 271:5777-5783 (1996)). One day post-transfection, the medium is changed and and cells are either stimulated with 1 ng/ml IL-1α or are left unstimulated. 12-16 hours post-stimulation, cells are washed twice with binding medium containing 5% (w/v) non-fat dry milk (5% MBM) and blocked with 2 ml of 5% MBM at room temperature for 30 minutes. Cells are then incubated at room temperature for 60-90 minutes with 1.5 ml/well of 5% MBM containing 1 μg/ml of an anti-IL-2Ra antibody (R&D Systems, Minneapolis, Minn.) with gentle rocking. Cells are washed once with 5% MBM and incubated with 1 μl/well of 5% MBM containing 1:100 dilution of 125I-goat anti-mouse IgG (Sigma, St. Louis, Mo.) for 60 minutes at room temperature. Wells are washed four times with 5% MBM and twice with phosphate-buffered saline. Wells are stripped by the addition of 1 ml of 0.5 M NaOH, and total counts are determined. Results are expressed as total CPM averged over two duplicate or three triplicate wells.

Thus, “a polypeptide having T1-R-like ligand III activity” includes polypeptides that exhibit T1-R ligand III protein activity in the above-described assay.

T1-R ligand III protein modulates immune system response to injury and infection in a dose-dependent manner in the above-described assay. Thus, “a polypeptide having T1-R ligand III protein activity” includes polypeptides that also exhibit any of the same immune system response to injury and infection activities in the above-described assays in a dose-dependent manner. Although the degree of dose-dependent activity need not be identical to that of the T1-R ligand III protein, preferably, “a polypeptide having T1-R ligand III protein activity” will exhibit substantially similar dose-dependence in a given activity as compared to the T1-R ligand III protein (i.e., the candidate polypeptide will exhibit greater activity or not more than about 25-fold less and, preferably, not more than about tenfold less activity relative to the reference T1-R ligand III protein).

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequence of the deposited cDNA or the nucleic acid sequence shown in FIG. I (SEQ ID NO:1) will encode a polypeptide “having T1-R ligand III protein activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having T1-R ligand III protein activity. This is because the skilled artisan is fully aware of amino acid substitutions that are either less likely or not likely to significantly effect protein function (e.g., replacing one aliphatic amino acid with a second aliphatic amino acid), as further described below.

Vectors and Host Cells

The present invention also relates to vectors which include the isolated DNA molecules of the present invention, host cells which are genetically engineered with the recombinant vectors, and the production of T1-R ligand III polypeptides or fragments thereof by recombinant techniques. The vector may be, for example, a phage, plasmid, viral or retroviral vector. Retroviral vectors may be replication competent or replication defective. In the latter case, viral propagation generally will occur only in complementing host cells.

The polynucleotides may be joined to a vector containing a selectable marker for propagation in a host. Generally, a plasmid vector is introduced in a precipitate, such as a calcium phosphate precipitate, or in a complex with a charged lipid. If the vector is a virus, it may be packaged in vitro using an appropriate packaging cell line and then transduced into host cells.

The DNA insert should be operatively linked to an appropriate promoter, such as the phage lambda PL promoter, the E coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination and, in the transcribed region, a ribosome binding site for translation. The coding portion of the transcripts expressed by the constructs will preferably include a translation initiating codon at the beginning and a termination codon (UAA, UGA or UAG) appropriately positioned at the end of the polypeptide to be translated.

As indicated, the expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells; insect cells such as Drosophila S2 and Spodoptera Sf9 cells; animal cells such as CHO, COS, 293 and Bowes melanoma cells; and plant cells. Appropriate culture mediums and conditions for the above-described host cells are known in the art.

Among vectors preferred for use in bacteria include pQE70, pQE60 and pQE-9, available from QIAGEN, Inc., supra; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Among preferred eukaryotic vectors are pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, (e.g. Davis et al., Basic Methods In Molecular Biology (1986)).

The polypeptide may be expressed in a modified form, such as a fusion protein, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties may be added to the polypeptide to facilitate purification. Such regions may be removed prior to final preparation of the polypeptide. The addition of peptide moieties to polypeptides to engender secretion or excretion, to improve stability and to facilitate purification, among others, are familiar and routine techniques in the art. A preferred fusion protein comprises a heterologous region from immunoglobulin that is useful to stabilize and purify proteins. For example, EP-A-O 464 533 (Canadian counterpart 2045869) discloses fusion proteins comprising various portions of constant region of immunoglobulin molecules together with another human protein or part thereof. In many cases, the Fc part in a fusion protein is thoroughly advantageous for use in therapy and diagnosis and thus results, for example, in improved pharmacokinetic properties (EP-A 0232 262). On the other hand, for some uses it would be desirable to be able to delete the Fc part after the fusion protein has been expressed, detected and purified in the advantageous manner described. This is the case when Fc portion proves to be a hindrance to use in therapy and diagnosis, for example when the fusion protein is to be used as antigen for immunizations. In drug discovery, for example, human proteins, such as hIL-5, have been fused with Fc portions for the purpose of high-throughput screening assays to identify antagonists of hIL-5 (see D. Bennett et al., J. Molec. Recog. 8:52-58 (1995) and K. Johanson et al., J. Biol. Chem. 270:9459-9471 (1995)).

The T1-R ligand III protein can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography (“HPLC”) is employed for purification. Polypeptides of the present invention include: products purified from natural sources, including bodily fluids, tissues and cells, whether directly isolated or cultured; products of chemical synthetic procedures; and products produced by recombinant techniques from a prokaryotic or eukaryotic host, including, for example, bacterial, yeast, higher plant, insect and mammalian cells. Depending upon the host employed in a recombinant production procedure, the polypeptides of the present invention may be glycosylated or may be non-glycosylated. In addition, polypeptides of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

### Polypeptides and Fragments

The invention further provides an isolated T1-R ligand III polypeptide having the amino acid sequence encoded by the deposited cDNA, or the amino acid sequence in SEQ ID NO:2, or the amino acid sequence in SEQ ID NO:4, or a peptide or polypeptide comprising a portion of the above polypeptides.

Variant and Mutant Polypeptides

To improve or alter the characteristics of T1-R ligand III polypeptides, protein engineering may be employed. Recombinant DNA technology known to those skilled in the art can be used to create novel mutant proteins or “muteins including single or multiple amino acid substitutions, deletions, additions or fusion proteins. Such modified polypeptides can show, e.g., enhanced activity or increased stability. In addition, they may be purified in higher yields and show better solubility than the corresponding natural polypeptide, at least under certain purification and storage conditions.

N-Terminal and C-Terminal Deletion Mutants

For instance, for many proteins, including the extracellular domain of a membrane associated protein or the mature form(s) of a secreted protein, it is known in the art that one or more amino acids may be deleted from the N-terminus or C-terminus without substantial loss of biological function. For instance, Ron and coworkers (J. Biol. Chem., 268:2984-2988 (1993)) reported modified keratinocyte growth factor proteins that had heparin binding activity even if 3, 8, or 27 amino-terminal amino acid residues were missing. In the present case, since the protein of the invention is a member of the IL-1RI-like polypeptide family, deletions of N-terminal amino acids up to the cysteine at position 15 of SEQ ID NO:2 and SEQ ID NO:4 may retain some biological activity such as modulation of the host immune system to injury and infection. Polypeptides having further N-terminal deletions including the cysteine residue at position 15 in SEQ ID NO:2 and SEQ ID NO:4 would not be expected to retain such biological activities because it is known that in many ligands cysteine residues are often required for forming a disulfide bridge to provide structural stability which is needed for receptor binding and signal transduction.

However, even if deletion of one or more amino acids from the N-terminus of a protein results in modification of loss of one or more biological functions of the protein, other biological activities may still be retained. Thus, the ability of the shortened protein to induce and/or bind to antibodies which recognize the complete or mature of the protein generally will be retained when less than the majority of the residues of the complete or mature protein are removed from the N-terminus. Whether a particular polypeptide lacking N-terminal residues of a complete protein retains such immunologic activities can readily be determined by routine methods described herein and otherwise known in the art.

Accordingly, the present invention further provides polypeptides having one or more residues deleted from the amino terminus of the amino acid sequence of the T1-R ligand III shown in SEQ ID NO:2 or in SEQ ID NO:4, up to the cysteine residue at position number 15, and polynucleotides encoding such polypeptides. In particular, the present invention provides polypeptides comprising the amino acid sequence of residues n-139 of SEQ ID NO:2 or residues n-191 of SEQ ID NO:4, where n is an integer in the range of −23 to +15, where +15 is the position of the first residue from the N-terminus of the complete T1-R ligand III polypeptide (shown in SEQ ID NO:2 or SEQ ID NO:4) believed to be required for modulation of the host immune system to injury and infection activity of the T1-R ligand III protein.

More in particular, the invention provides polynucleotides encoding polypeptides having the amino acid sequence of residues of −24-139, −23-139, −22-139, −21-139, −20-139, −19-139, −18-139, −17-139, −16-139, −15-139, −14-139, −13-139, −12-139, −11-139, −10-139, −9-139, −8-139, −7-139, −6-139, −5-139, −4-139, −3-139, −2-139, −1-139, 1-139, 2-139, 3-139, 4-139, 5-139, 6-139, 7-139, 8-139, 9-139, 10-139, 11-139, 12-139, 13-139, 14-139, 15-139, of SEQ ID NO:2 or −24-191, −23-191, −22-191, −21-191, −20-191, −19-191, −18-191, −17-191, −16-191, −15-191, −14-191, −13-191, −12-191, −11-191, −10-191, −9-191, −8-191, −7-191, −6-191, −5-191, −4-191, −3-191, −2-191, −1-191, 1-191, 2-191, 3-191, 4-191, 5-191, 6-191, 7-191, 8-191, 9-191, 10-191, 11-191, 12-191, 13-191, 14-191, 15-191, of SEQ ID NO:4. Polynucleotides encoding these polypeptides also are provided.

Similarly, many examples of biologically functional C-terminal deletion muteins are known. For instance, Interferon gamma shows up to ten times higher activities by deleting 8-10 amino acid residues from the carboxy terminus of the protein (Dobeli, et al., J. Biotechnol. 7:199-216 (1988)). In the present case, deletions of C-terminal amino acids up to about 10 additional C -terminal residues (i.e., up to the lysine residue at position 129 of SEQ ID NO:2 or up to the lysine at position 181 of SEQ ID NO:4) may retain some biological activity such as modulation of the host immune system to injury and infection. Polypeptides having further C-terminal deletions including the lysine residue at position 129 of SEQ ID NO:2 or position 181 of SEQ ID NO:4 would not be expected to retain such biological activities because it is thought that this residue may be the beginning of a conserved domain required for biological activities.

However, even if deletion of one or more amino acids from the C-terminus of a protein results in modification of loss of one or more biological functions of the protein, other biological activities may still be retained. Thus, the ability of the shortened protein to induce and/or bind to antibodies which recognize the complete or mature form of the protein generally will be retained when less than the majority of the residues of the complete or mature form of the protein are removed from the C-terminus. Whether a particular polypeptide lacking C-terminal residues of a complete protein retains such immunologic activities can readily be determined by routine methods described herein and otherwise known in the art.

Accordingly, the present invention further provides polypeptides having one or more residues from the carboxy terminus of the amino acid sequence of the T1-R ligand III shown in SEQ ID NO:2, up to the lysine residue at position 129 of SEQ ID NO:2 or position 181 of SEQ ID NO:4, and polynucleotides encoding such polypeptides. In particular, the present invention provides polypeptides having the amino acid sequence of residues -23-m of the amino acid sequence in SEQ ID NO:2, where m is any integer in the range of 153 to 162, and residue 153 is the position of the first residue from the C-terminus of the complete T1-R ligand III polypeptide (shown in SEQ ID NO:2) believed to be required for modulation of the host immune system to injury and infection of the T1-R ligand III protein. The present invention also provides polypeptides having the amino acid sequence of residues −23-m of the amino acid sequence in SEQ ID NO:4, where m is any integer in the range of 181 to 190, and residue 181 is the position of the first residue from the C-terminus of the complete T1-R ligand III polypeptide (shown in SEQ ID NO:4) believed to be required for modulation of the host immune system to injury and infection of the T1-R ligand III protein.

More in particular, the invention provides polynucleotides encoding polypeptides having the amino acid sequence of residues −23-153, −23-154, −23-155, −23-156, −23-157, −23-158, −23-159, −23-160, −23-161, −23-162, of SEQ ID NO:2. Polynucleotides encoding these polypeptides also are provided. The invention provides polynucleotides encoding polypeptides having the amino acid sequence of residues −23-181, −23-182, −23-183, −23-184, −23-185, −23-186, −23-187, −23-188, −23-189, −23-190, of SEQ ID NO:4. Polynucleotides encoding these polypeptides also are provided.

The invention also provides polypeptides having one or more amino acids deleted from both the amino and the carboxyl termini, which may be described generally as having residues n-m of SEQ ID NO:2 or of SEQ ID NO:4, where n and m are integers as described above.

Also included are a nucleotide sequence encoding a polypeptide consisting of a portion of the complete T1-R ligand III amino acid sequence encoded by the cDNA clone contained in ATCC Deposit No. 97859 or in ATCC Deposit No. 97858, where this portion excludes from 1 to about 38 amino acids from the amino terminus of the complete amino acid sequence encoded by the cDNA clone contained in ATCC Deposit No. 97859 or in ATCC Deposit No. 97858, or from 1 to about 10 amino acids from the carboxy terminus, or any combination of the above amino terminal and carboxy terminal deletions, of the complete amino acid sequence encoded by the cDNA clone contained in ATCC Deposit No. 97859 or in ATCC Deposit No. 97858. Polynucleotides encoding all of the above deletion mutant polypeptide forms also are provided.

Other Mutants

In addition to terminal deletion forms of the protein discussed above, it also will be recognized by one of ordinary skill in the art that some amino acid sequences of the T1-R ligand III polypeptide can be varied without significant effect of the structure or function of the protein. If such differences in sequence are contemplated, it should be remembered that there will be critical areas on the protein which determine activity.

Thus, the invention further includes variations of the T1-R ligand III polypeptide which show substantial T1-R ligand III polypeptide activity or which include regions of T1-R ligand III protein such as the protein portions discussed below. Such mutants include deletions, insertions, inversions, repeats, and type substitutions selected according to general rules known in the art so as have little effect on activity. For example, guidance concerning how to make phenotypically silent amino acid substitutions is provided by Bowie and colleagues (“Deciphering the Message in Protein Sequences: Tolerance to Amino Acid Substitutions,” Science 247:1306-1310 (1990)), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality.

As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described (Bowie, J. U. et al., supra, and the references cited therein). Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

Thus, the fragment, derivative or analog of the polypeptide of SEQ ID NO:2, or that encoded by the deposited cDNA, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the above form of the polypeptide, such as an IgG Fc fusion region peptide or leader or secretory sequence or a sequence which is employed for purification of the above form of the polypeptide or a proprotein sequence. Such fragments, derivatives and analogs are deemed to be within the scope of those skilled in the art from the teachings herein.

Thus, the T1-R ligand III of the present invention may include one or more amino acid substitutions, deletions or additions, either from natural mutations or human manipulation. As indicated, changes are preferably of a minor nature, such as conservative amino acid substitutions that do not significantly affect the folding or activity of the protein (see Table 1).

TABLE 1
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Amino acids in the T1-R ligand III protein of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as receptor binding or in vitro or in vitro proliferative activity.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic (Pinckard et al., Clin. Exp. Immunol. 2:331-340 (1967); Robbins et al., Diabetes 36: 838-845 (1987); Cleland et al., Crit. Rev. Ther. Drug Carrier Sys. 10:307-377 (1993)).

Replacement of amino acids can also change the selectivity of the binding of a ligand to cell surface receptors. For example, Ostade and coworkers (Nature 361:266-268 (1993)) describe certain mutations resulting in selective binding of TNF-α to only one of the two known types of TNF receptors. Sites that are critical for ligand-receptor binding can also be determined by structural analysis such as crystallization, nuclear magnetic resonance or photoaffinity labeling (Smith et al., J. Mol. Biol. 224:899-904 (1992) and de Vos et al. Science 255:306-312 (1992)).

The polypeptides of the present invention are preferably provided in an isolated form, and preferably are substantially purified. A recombinantly produced version of the T1-R ligand III polypeptide can be substantially purified by the one-step method described by Smith and Johnson (Gene 67:31-40 (1988)). Polypeptides of the invention also can be purified from natural or recombinant sources using anti-T1-R ligand III antibodies of the invention in methods which are well known in the art of protein purification.

Further polypeptides of the present invention include polypeptides which have at least 90% similarity, more preferably at least 95% similarity, and still more preferably at least 96%, 97%, 98% or 99% similarity to those described above. The polypeptides of the invention also comprise those which are at least 80% identical, more preferably at least 90% or 95% identical, still more preferably at least 96%, 97%, 98% or 99% identical to the polypeptide encoded by the deposited cDNAs or to the polypeptide of SEQ ID NO:2 or SEQ ID NO:4, and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, WI 53711) and the default settings for determining similarity. Bestfit uses the local homology algorithm of Smith and Waterman (Advances in Applied Mathematics 2:482-489, 1981) to find the best segment of similarity between two sequences.

By a polypeptide having an amino acid sequence at least, for example, 95% “identical” to a reference amino acid sequence of a T1-R ligand III polypeptide is intended that the amino acid sequence of the polypeptide is identical to the reference sequence except that the polypeptide sequence may include up to five amino acid alterations per each 100 amino acids of the reference amino acid of the T1-R ligand III polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in SEQ ID NO:2 or in SEQ ID NO:4 or to the amino acid sequence encoded by deposited cDNA clones can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptide of the present invention could be used as a molecular weight marker on SDS-PAGE gels or on molecular sieve gel filtration columns using methods well known to those of skill in the art.

As described in detail below, the polypeptides of the present invention can also be used to raise polyclonal and monoclonal antibodies, which are useful in assays for detecting T1-R ligand III protein expression as described below or as agonists and antagonists capable of enhancing or inhibiting T1-R ligand III protein function. Further, such polypeptides can be used in the yeast two-hybrid system to “capture” T1-R ligand III protein binding proteins which are also candidate agonists and antagonists according to the present invention. The yeast two hybrid system is described by Fields and Song (Nature 340:245-246 (1989)).

Epitope-Bearing Portions

In another aspect, the invention provides a peptide or polypeptide comprising an epitope-bearing portion of a polypeptide of the invention. The epitope of this polypeptide portion is an immunogenic or antigenic epitope of a polypeptide of the invention. An “immunogenic epitope” is defined as a part of a protein that elicits an antibody response when the whole protein is the immunogen. On the other hand, a region of a protein molecule to which an antibody can bind is defined as an “antigenic epitope.” The number of immunogenic epitopes of a protein generally is less than the number of antigenic epitopes (Geysen et al., Proc. Natl. Acad. Sci. USA 81:3998-4002 (1983)).

As to the selection of peptides or polypeptides bearing an antigenic epitope (i.e., that contain a region of a protein molecule to which an antibody can bind), it is well known in that art that relatively short synthetic peptides that mimic part of a protein sequence are routinely capable of eliciting an antiserum that reacts with the partially mimicked protein (Sutcliffe, J. G., et al., “Antibodies that react with predetermined sites on proteins,” Science, 219:660-666 (1983)). Peptides capable of eliciting protein-reactive sera are frequently represented in the primary sequence of a protein, can be characterized by a set of simple chemical rules, and are confined neither to immunodominant regions of intact proteins (i.e., immunogenic epitopes) nor to the amino or carboxyl terminals. Antigenic epitope-bearing peptides and polypeptides of the invention are therefore useful to raise antibodies, including monoclonal antibodies, that bind specifically to a polypeptide of the invention (Wilson et al., Cell 37:767-778 (1984)).

Antigenic epitope-bearing peptides and polypeptides of the invention preferably contain a sequence of at least seven, more preferably at least nine and most preferably between about 15 to about 30 amino acids contained within the amino acid sequence of a polypeptide of the invention. Non-limiting examples of antigenic polypeptides or peptides that can be used to generate T1-R ligand 111-specific antibodies include: a polypeptide comprising amino acid residues from about 1-10, 30-40, 45-57, 89-100, 120-132, 150-162 of SEQ ID NO:2 or a polypeptide comprising amino acid residues from about 1-10, 30-40, 45-57, 89-100, 120-132, 155-165, and 201-212 of SEQ ID NO:4. These polypeptide fragments have been determined to bear antigenic epitopes of the T1-R ligand III protein by the analysis of the Jameson-Wolf antigenic index, as shown in FIG. 3A and FIG. 3B, above.

The epitope-bearing peptides and polypeptides of the invention may be produced by any conventional means (Houghten, R. A. “General method for the rapid solid-phase synthesis of large numbers of peptides: specificity of antigen-antibody interaction at the level of individual amino acids.” Proc. Natl. Acad. Sci. USA 82:5131-5135 (1985); this “Simultaneous Multiple Peptide Synthesis (SMPS)” process is further described in U.S. Pat. No. 4,631,211 to Houghten and colleagues (1986).

Epitope-bearing peptides and polypeptides of the invention are used to induce antibodies according to methods well known in the art. See, for instance, Sutcliffe et al., supra; Wilson et al., supra; Chow, M. et al., Proc. Natl. Acad. Sci. USA 82:910-914; and Bittle, F. J. et al., J. Gen. Virol. 66:2347-2354 (1985). Immunogenic epitope-bearing peptides of the invention, i.e., those parts of a protein that elicit an antibody response when the whole protein is the immunogen, are identified according to methods known in the art. See, for instance, Geysen et al., supra. Further still, U.S. Pat. No. 5,194,392 to Geysen (1990) describes a general method of detecting or determining the sequence of monomers (amino acids or other compounds) which is a topological equivalent of the epitope (i.e., a “mimotope”) which is complementary to a particular paratope (antigen binding site) of an antibody of interest. More generally, U.S. Pat. No. 4,433,092 to Geysen (1989) describes a method of detecting or determining a sequence of monomers which is a topographical equivalent of a ligand which is complementary to the ligand binding site of a particular receptor of interest. Similarly, U.S. Pat. No. 5,480,971 to Houghten, R. A. et al. (1996) on Peralkylated Oligopeptide Mixtures discloses linear Cl-C7-alkyl peralkylated oligopeptides and sets and libraries of such peptides, as well as methods for using such oligopeptide sets and libraries for determining the sequence of a peralkylated oligopeptide that preferentially binds to an acceptor molecule of interest. Thus, non-peptide analogs of the epitope-bearing peptides of the invention also can be made routinely by these methods. Fusion Proteins As one of skill in the art will appreciate, T1-R ligand III polypeptides of the present invention and the epitope-bearing fragments thereof described above can be combined with parts of the constant domain of immunoglobulins (IgG), resulting in chimeric polypeptides. These fusion proteins facilitate purification and show an increased half-life in vivo. This has been shown, e.g., for chimeric proteins consisting of the first two domains of the human CD4-polypeptide and various domains of the constant regions of the heavy or light chains of mammalian immunoglobulins (EP A 394,827; Traunecker et al., Nature 331:84-86 (1988)). Fusion proteins that have a disulfide-linked dimeric structure due to the IgG part can also be more efficient in binding and neutralizing other molecules than the monomeric T1-R ligand III protein or protein fragment alone (Fountoulakis et al., J. Biochem. 270:3958-3964 (1995)).

Antibodies

T1-R ligand III-protein specific antibodies for use in the present invention can be raised against the intact T1-R ligand III protein or an antigenic polypeptide fragment thereof, which may be presented together with a carrier protein, such as an albumin, to an animal system (such as rabbit or mouse) or, if it is long enough (at least about 25 amino acids), without a carrier.

As used herein, the term “antibody” (Ab) or “monoclonal antibody” (Mab) is meant to include intact molecules as well as antibody fragments (such as, for example, Fab and F(ab′)2 fragments) which are capable of specifically binding to T1-R ligand III protein. Fab and F(ab′)2 fragments lack the Fc fragment of intact antibody, clear more rapidly from the circulation, and may have less non-specific tissue binding of an intact antibody (Wahl et al., J. Nucl. Med. 24:316-325 (1983)). Thus, these fragments are preferred.

The antibodies of the present invention may be prepared by any of a variety of methods. For example, cells expressing the T1-R ligand III protein or an antigenic fragment thereof can be administered to an animal in order to induce the production of sera containing polyclonal antibodies. In a preferred method, a preparation of T1-R ligand III protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or T1-R ligand III protein binding fragments thereof). Such monoclonal antibodies can be prepared using hybridoma technology (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., (1981) pp. 563-681). In general, such procedures involve immunizing an animal (preferably a mouse) with a T1-R ligand III protein antigen or, more preferably, with a T1-R ligand III protein-expressing cell. Suitable cells can be recognized by their capacity to bind anti-T1-R ligand III protein antibody. Such cells may be cultured in any suitable tissue culture medium; however, it is preferable to culture cells in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin. The splenocytes of such mice are extracted and fused with a suitable myeloma cell line. Any suitable myeloma cell line may be employed in accordance with the present invention; however, it is preferable to employ the parent myeloma cell line (SP20), available from the American Type Culture Collection, Rockville, Maryland. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the T1-R ligand III protein antigen.

Alternatively, additional antibodies capable of binding to the T1-R ligand III protein antigen may be produced in a two-step procedure through the use of anti-idiotypic antibodies. Such a method makes use of the fact that antibodies are themselves antigens, and that, therefore, it is possible to obtain an antibody which binds to a second antibody. In accordance with this method, T1-R ligand III-protein specific antibodies are used to immunize an animal, preferably a mouse. The splenocytes of such an animal are then used to produce hybridoma cells, and the hybridoma cells are screened to identify clones which produce an antibody whose ability to bind to the T1-R ligand III protein-specific antibody can be blocked by the T1-R ligand III protein antigen. Such antibodies comprise anti-idiotypic antibodies to the T1-R ligand III protein-specific antibody and can be used to immunize an animal to induce formation of further T1-R ligand III protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, T1-R ligand III protein-binding fragments can be produced through the application of recombinant DNA technology or through synthetic chemistry.

For in vivo use of anti-T1-R ligand III in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).

### Immune System-Related Disorders

Diagnosis

The present inventors have discovered that T1-R ligand III is expressed in embryonic and endometrial tumor tissues. For a number of immune system-related disorders, substantially altered (increased or decreased) levels of T1-R ligand III gene expression can be detected in immune system tissue or other cells or bodily fluids (e.g., sera, plasma, urine, synovial fluid or spinal fluid) taken from an individual having such a disorder, relative to a “standard” T1-R ligand III gene expression level, that is, the T1-R ligand III expression level in immune system tissues or bodily fluids from an individual not having the immune system disorder. Thus, the invention provides a diagnostic method useful during diagnosis of a immune system disorder, which involves measuring the expression level of the gene encoding the T1-R ligand III protein in immune system tissue or other cells or body fluid from an individual and comparing the measured gene expression level with a standard T1-R ligand III gene expression level, whereby an increase or decrease in the gene expression level compared to the standard is indicative of an immune system disorder.

In particular, it is believed that certain tissues in mammals with cancer of the immune system express significantly alterred levels of the T1-R ligand III protein and mRNA encoding the T1-R ligand III protein when compared to a corresponding “standard” level. Further, it is believed that alterred levels of the T1-R ligand III protein can be detected in certain body fluids (e.g., sera, plasma, urine, and spinal fluid) from mammals with such a cancer when compared to sera from mammals of the same species not having the cancer.

Thus, the invention provides a diagnostic method useful during diagnosis of an immune system disorder, including cancers of this system, which involves measuring the expression level of the gene encoding the T1-R ligand III protein in immune system tissue or other cells or body fluid from an individual and comparing the measured gene expression level with a standard T1-R ligand III gene expression level, whereby an increase or decrease in the gene expression level compared to the standard is indicative of an immune system disorder.

Where a diagnosis of a disorder in the immune system including diagnosis of a tumor, has already been made according to conventional methods, the present invention is useful as a prognostic indicator, whereby patients exhibiting enhanced or depressed T1-R ligand III gene expression will experience a worse clinical outcome relative to patients expressing the gene at a level nearer the standard level.

By “assaying the expression level of the gene encoding the T1-R ligand III protein” is intended qualitatively or quantitatively measuring or estimating the level of the T1-R ligand III protein or the level of the mRNA encoding the T1-R ligand III protein in a first biological sample either directly (e.g., by determining or estimating absolute protein level or mRNA level) or relatively (e.g., by comparing to the T1-R ligand III protein level or mRNA level in a second biological sample). Preferably, the T1-R ligand III protein level or mRNA level in the first biological sample is measured or estimated and compared to a standard T1-R ligand III protein level or mRNA level, the standard being taken from a second biological sample obtained from an individual not having the disorder or being determined by averaging levels from a population of individuals not having a disorder of the immune system. As will be appreciated in the art, once a standard T1-R ligand III protein level or mRNA level is known, it can be used repeatedly as a standard for comparison.

By “biological sample” is intended any biological sample obtained from an individual, body fluid, cell line, tissue culture, or other source which contains T1-R ligand III protein or mRNA. As indicated, biological samples include body fluids (such as sera, plasma, urine, synovial fluid and spinal fluid) which contain free T1-R ligand III protein, immune system tissue, and other tissue sources found to express complete or mature T1-R ligand III or a T1-R ligand III receptor. Methods for obtaining tissue biopsies and body fluids from mammals are well known in the art. Where the biological sample is to include mRNA, a tissue biopsy is the preferred source.

The present invention is useful for diagnosis or treatment of various immune system-related disorders in mammals, preferably humans. Such disorders include any disregulation of immune cell function including, but not limited to, leukemia, atheroscerosis, autoimmune disease, inflammation, metabolic dysfunction, immune-mediated diseases and the like.

Total cellular RNA can be isolated from a biological sample using any suitable technique such as the single-step guanidinium-thiocyanate-phenol-chloroform method described in Chomczynski and Sacchi, Anal. Biochem. 162:156-159 (1987). Levels of mRNA encoding the T1-R ligand III protein are then assayed using any appropriate method. These include Northern blot analysis, S1 nuclease mapping, the polymerase chain reaction (PCR), reverse transcription in combination with the polymerase chain reaction (RT-PCR), and reverse transcription in combination with the ligase chain reaction (RT-LCR).

Assaying T1-R ligand III protein levels in a biological sample can occur using antibody-based techniques. For example, T1-R ligand III protein expression in tissues can be studied with classical immunohistological methods (Jalkanen, M., et al., J. Cell. Biol. 101:976-985 (1985); Jalkanen, M., et al., J. Cell . Biol. 105:3087-3096 (1987)). Other antibody-based methods useful for detecting T1-R ligand III protein gene expression include immunoassays, such as the enzyme linked immunosorbent assay (ELISA) and the radioimmunoassay (RIA). Suitable antibody assay labels are known in the art and include enzyme labels, such as, glucose oxidase, and radioisotopes, such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin.

In addition to assaying T1-R ligand III protein levels in a biological sample obtained from an individual, T1-R ligand III protein can also be detected in vivo by imaging. Antibody labels or markers for in vivo imaging of T1-R ligand III protein include those detectable by X-radiography, NMR or ESR. For X-radiography, suitable labels include radioisotopes such as barium or cesium, which emit detectable radiation but are not overtly harmful to the subject. Suitable markers for NMR and ESR include those with a detectable characteristic spin, such as deuterium, which may be incorporated into the antibody by labeling of nutrients for the relevant hybridoma.

A T1-R ligand III protein-specific antibody or antibody fragment which has been labeled with an appropriate detectable imaging moiety, such as a radioisotope (for example, ¹³¹I, ¹¹²In, ⁹⁹mTc), a radio-opaque substance, or a material detectable by nuclear magnetic resonance, is introduced (for example, parenterally, subcutaneously or intraperitoneally) into the mammal to be examined for immune system disorder. It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. The labeled antibody or antibody fragment will then preferentially accumulate at the location of cells which contain T1-R ligand III protein. In vivo tumor imaging is described in S. W. Burchiel et al., “Immunopharmacokinetics of Radiolabeled Antibodies and Their Fragments” (Chapter 13 in Tumor Imaging: The Radiochemical Detection of Cancer, S. W. Burchiel and B. A. Rhodes, eds., Masson Publishing Inc. (1982)).

Treatment

As noted above, T1-R ligand III polynucleotides and polypeptides are useful for diagnosis of conditions involving abnormally high or low expression of T1-R ligand III activities. Given the cells and tissues where T1-R ligand III is expressed as well as the activities modulated by T1-R ligand III, it is readily apparent that a substantially altered (increased or decreased) level of expression of T1-R ligand III in an individual compared to the standard or “normal” level produces pathological conditions related to the bodily system(s) in which T1-R ligand III is expressed and/or is active.

It will also be appreciated by one of ordinary skill that, since the T1-R ligand III protein of the invention is a member of the IL-1RBP family the mature secreted form of the protein may be released in soluble form from the cells which express the T1-R ligand III by proteolytic cleavage. Therefore, when T1-R ligand III mature form is added from an exogenous source to cells, tissues or the body of an individual, the protein will exert its physiological activities on its target cells of that individual.

Therefore, it will be appreciated that conditions caused by a decrease in the standard or normal level of T1-R ligand III activity in an individual, particularly disorders of the immune system, can be treated by administration of T1-R ligand III polypeptide (in the form of a mature protein). Thus, the invention also provides a method of treatment of an individual in need of an increased level of T1-R ligand III activity comprising administering to such an individual a pharmaceutical composition comprising an amount of an isolated T1-R ligand III polypeptide of the invention, particularly a mature form of the T1-R ligand III protein of the invention, effective to increase the T1-R ligand III activity level in such an individual.

Formulations

The T1-R ligand III polypeptide composition will be formulated and dosed in a fashion consistent with good medical practice, taking into account the clinical condition of the individual patient (especially the side effects of treatment with T1-R ligand III polypeptide alone), the site of delivery of the T1-R ligand III polypeptide composition, the method of administration, the scheduling of administration, and other factors known to practitioners. The “effective amount” of T1-R ligand III polypeptide for purposes herein is thus determined by such considerations.

As a general proposition, the total pharmaceutically effective amount of T1-R ligand III polypeptide administered parenterally per dose will be in the range of about 1 μg/kg/day to 10 mg/kg/day of patient body weight, although, as noted above, this will be subject to therapeutic discretion. More preferably, this dose is at least 0.01 mg/kg/day, and most preferably for humans between about 0.01 and 1 mg/kg/day for the hormone. If given continuously, the T1-R ligand III polypeptide is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions containing the T1-R ligand III of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrastemal, subcutaneous and intra-articular injection and infusion.

The T1-R ligand III polypeptide is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release T1-R ligand III polypeptide compositions also include liposomally entrapped T1-R ligand III polypeptide. Liposomes containing T1-R ligand III polypeptide are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal T1-R ligand III polypeptide therapy.

For parenteral administration, in one embodiment, the T1-R ligand III polypeptide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to polypeptides.

Generally, the formulations are prepared by contacting the T1-R ligand III polypeptide uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes.

The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The T1-R ligand III polypeptide is typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation of T1-R ligand III polypeptide salts.

T1-R ligand III polypeptide to be used for therapeutic administration must be sterile. Sterility is readily accomplished by filtration through sterile filtration membranes (e.g., 0.2 micron membranes). Therapeutic T1-R ligand III polypeptide compositions generally are placed into a container having a sterile access port, for example, an intravenous solution bag or vial having a stopper pierceable by a hypodermic injection needle.

T1-R ligand III polypeptide ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous T1-R ligand III polypeptide solution, and the resulting mixture is lyophilized. The infusion solution is prepared by reconstituting the lyophilized T1-R ligand III polypeptide using bacteriostatic Water-for-Injection.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the polypeptides of the present invention may be employed in conjunction with other therapeutic compounds.

Agonists and Antagonists—Assays and Molecules

The invention also provides a method of screening compounds to identify those which enhance or block the action of T1-R ligand III on cells, such as its interaction with T1-R ligand III-binding molecules such as receptor molecules. An agonist is a compound which increases the natural biological functions of T1-R ligand III or which functions in a manner similar to T1-R ligand III, while antagonists decrease or eliminate such functions.

In another aspect of this embodiment the invention provides a method for identifying a receptor protein or other ligand-binding protein which binds specifically to a T1-R ligand III polypeptide. For example, a cellular compartment, such as a membrane or a preparation thereof, may be prepared from a cell that expresses a molecule that binds T1-R ligand III. The preparation is incubated with labeled T1-R ligand III and complexes of T1-R ligand III bound to the receptor or other binding protein are isolated and characterized according to routine methods known in the art. Alternatively, the T1-R ligand III polypeptide may be bound to a solid support so that binding molecules solubilized from cells are bound to the column and then eluted and characterized according to routine methods.

In the assay of the invention for agonists or antagonists, a cellular compartment, such as a membrane or a preparation thereof, may be prepared from a cell that expresses a molecule that binds T1-R ligand III, such as a molecule of a signaling or regulatory pathway modulated by T1-R ligand III. The preparation is incubated with labeled T1-R ligand III in the absence or the presence of a candidate molecule which may be a T1-R ligand III agonist or antagonist. The ability of the candidate molecule to bind the binding molecule is reflected in decreased binding of the labeled ligand. Molecules which bind gratuitously, i.e., without inducing the effects of T1-R ligand III on binding the T1-R ligand III binding molecule, are most likely to be good antagonists. Molecules that bind well and elicit effects that are the same as or closely related to T1-R ligand III are agonists.

T1-R ligand III-like effects of potential agonists and antagonists may by measured, for instance, by determining activity of a second messenger system following interaction of the candidate molecule with a cell or appropriate cell preparation, and comparing the effect with that of T1-R ligand III or molecules that elicit the same effects as T1-R ligand III. Second messenger systems that may be useful in this regard include but are not limited to AMP guanylate cyclase, ion channel or phosphoinositide hydrolysis second messenger systems.

Another example of an assay for T1-R ligand III antagonists is a competitive assay that combines T1-R ligand III and a potential antagonist with membrane-bound T1-R ligand III receptor molecules or recombinant T1-R ligand III receptor molecules under appropriate conditions for a competitive inhibition assay. T1-R ligand III can be labeled, such as by radioactivity, such that the number of T1-R ligand III molecules bound to a receptor molecule can be determined accurately to assess the effectiveness of the potential antagonist.

Potential antagonists include small organic molecules, peptides, polypeptides and antibodies that bind to a polypeptide of the invention and thereby inhibit or extinguish its activity. Potential antagonists also may be small organic molecules, a peptide, a polypeptide such as a closely related protein or antibody that binds the same sites on a binding molecule, such as a receptor molecule, without inducing T1-R ligand III-induced activities, thereby preventing the action of T1-R ligand III by excluding T1-R ligand III from binding.

By the invention, disorders caused by enhanced levels of T1-R ligand III protein activity can be treated by administering an effective amount of an antagonist of a T1-R ligand III polypeptide of the invention. Therefore, antibodies (preferably monoclonal) or antibody fragments that bind a T1-R ligand III polypeptide of the present invention are useful in treating T1-R ligand III-related disorders as are soluble fragments of T1-R ligand III proteins which compete with the intact protein for binding to the T1-R ligand III receptor. Such antibodies and/or soluble fragments of T1-R ligand III proteins are preferably provided in pharmaceutically acceptable compositions.

The pharmaceutical compositions of the present invention may be administered, for example, by the parenteral, subcutaneous, intravenous, intramuscular, intraperitoneal, transdermal, or buccal routes. Alternatively, or concurrently, administration may be oral. The dosage administered will be dependent upon the age, health, and weight of the recipient, kind of concurrent treatment, if any, frequency of treatment, and the nature of the effect desired.

Compositions within the scope of this invention include all compositions wherein the antibody, fragment or derivative is contained in an amount effective to achieve its intended purpose. While individual needs vary, determination of optimal ranges of effective amounts of each component is within the skill of the art. The effective dose is a function of the individual chimeric or monoclonal antibody, the presence and nature of a conjugated therapeutic agent (see below), the patient and his clinical status, and can vary from about 10 ng/kg body weight to about 100 mg/kg body weight. The preferred dosages comprise 0.1 to 10 mg/kg body wt.

Preparations of an T1-R ligand III antibody or fragment for parenteral administration, such as in detectably labeled form for imaging or in a free or conjugated form for therapy, include sterile aqueous or non-aqueous solutions, suspensions, and emulsions. Examples of non-aqueous solvents are propyleneglycol, polyethyleneglycol, vegetable oil such as olive oil, and injectable organic esters such as ethyloleate. Aqueous carriers include water, alcoholic/aqueous solutions, emulsions or suspensions, including saline and buffered media, parenteral vehicles including sodium chloride solution, Ringer's dextrose, dextrose and sodium chloride, lactated Ringer's, or fixed oils. Intravenous vehicles include fluid and nutrient replenishers, such as those based on Ringer's dextrose, and the like. Preservatives and other additives may also be present, such as, for example, antimicrobials, anti-oxidants, chelating agents, and inert gases and the like. See, generally, Remington's Pharmaceutical Science, 16th ed., Mack Publishing Co., Easton, Pa., 19801.

The antibodies described herein may be advantageously utilized in combination with other monoclonal or chimeric antibodies, or with lymphokines or hemopoietic growth factors, etc., which serve to increase the number or activity of effector cells which interact with the antibodies.

Other potential antagonists include antisense molecules. Antisense technology can be used to control gene expression through antisense DNA or RNA or through triple-helix formation. Antisense techniques are discussed, for example, in Okano, J. Neurochem. 56: 560 (1991); “Oligodeoxynucleotides as Antisense Inhibitors of Gene Expression.” CRC Press, Boca Raton, FL (1988). Triple helix formation is discussed in, for instance Lee et al., Nucleic Acids Research 6:3073 (1979); Cooney et al., Science 241:456 (1988); and Dervan et al., Science 251:1360 (1991). The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide is designed to be complementary to a region of the gene involved in transcription thereby preventing transcription and the production of T1-R ligand III. The antisense RNA oligonucleotide hybridizes to the mRNA in vivo and blocks translation of the mRNA molecule into T1-R ligand III polypeptide. The oligonucleotides described above can also be delivered to cells such that the antisense RNA or DNA may be expressed in vivo to inhibit production of T1-R ligand III protein.

The agonists and antagonists may be employed in a composition with a pharmaceutically acceptable carrier, e.g., as described above. The antagonists may be employed for instance to inhibit T1-R ligand III activity to treat cancer, autoimmune diseases, and inflammatory diseases. Any of the above antagonists may be employed in a composition with a pharmaceutically acceptable carrier, e.g., as hereinafter described.

The T1-R ligand III polypeptides of the present invention are expected to have pleiotropic biological effects including many of those shown in Table 2 below. Similar biological effects have been shown for IL-1, particularly those associated with pancreatic endocrine tissue (Mandrup-Poulsen, T., et al., Cytokine 5:185 (1993)), thyroid glands (Rasmussen, A.K., Autoimmunity 16:141 (1993)), hypothalamic-pituitary-adrenal axis (Fantuzzi, G., & Ghezzi, P., Mediator Inflamm. 2:263 (1993); Rivier, C., Ann. NY Acad. Sci. 697:97 (1993); Rivier, C., & Rivest, S., Ciba. Found. Symp. 172:204 (1993)), fever (Coceani, F., “Fever: Basic Mechanisms and Management”, New York, N.Y., Raven (1991) p. 59), bone metabolism (Tatakis, D. N., J. Peridontol 64:416 (1993)), destruction of cartilage in the pathogenesis of rheumatoid arthritis (Arend, W.P., & Dayer, J.M., Arthritis Rheum 33:305 (1990); Krane, S. M., et al., Ann. NY Acad. Sci. 580:340 (1990)), uterine implantation (Lewis, M. P., et al., Placenta 15:13 (1994)), and loss of lean body mass (Roubenoff, R., et al., J. Clin. Invest. 93:2379 (1994)).

TABLE 2
POSSIBLE BIOLOGIC EFFECTS OF T1-R LIGAND III

EFFECTS OF SYSTEMICALLY INJECTED T1-R LIGAND III

```
FEVER; INCREASED SLOW WAVE SLEEP; SOCIAL DEPRESSION; ANOREXIA
HYPOTENSION; MYOCARDIAL SUPPRESSION; TACHYCARDIA; LACTIC
  ACIDOSIS
INCREASED CIRCULATING NITRIC OXIDE; HYPOAMINOACIDEMIA
HYPERINSULINEMIA; HYPERGLYCEMIA; HYPOGLYCEMIA
STIMULATION OF HYPOTHALAMIC-PITUITARY-ADRENAL AXIS
RELEASE OF HYPOTHALAMIC MONOAMINES AND NEUROPEPTIDES
NEUTROPHILIA; INCREASED MARROW CELLULARITY; INCREASED
  PLATELETS
INCREASED HEPATIC ACUTE PHASE PROTEIN SYNTHESIS
HYPOFERREMIA; HYPOZINCEMIA; INCREASED SODIUM EXCRETION
HYPERLIPIDEMIA; INCREASED MUSCLE PROTEIN BREAKDOWN
HYPOALBUMINEMIA; DECREASED DRUG METABOLISM
INCREASED METASTASES
INCREASED NONSPECIFIC RESISTANCE TO INFECTION (PRETREATMENT)
LEARNING DEFECTS IN OFFSPRING AFTER MATERNAL IL-1 TREATMENT
```

EFFECTS OF LOCALLY INJECTED T1-R LIGAND III

```
INFILTRATION OF NEUTROPHILIS INTO RABBITS KNEE JOINT
INCREASED PROTEOGLYCAN BREAKDOWN IN RABBIT KNEE JOINT
INDUCTION OF UVEITIS FOLLOWING INTRAVITREAL INJECTION
ANGIOGENESIS IN ANTERIOR CHAMBER OF EYE
CELLULAR INFILTRATE AND CYTOKINE INDUCTION IN CEREBRAL
  VENTRICLES
NEUTROPHIL AND ALBUMIN INFLUX INTO LUNGS AFTER INTRATRACHEAL
  INSTILLATION
```

CHANGES IN IMMUNOLOGIC RESPONSES

INCREASED ANTIBODY PRODUCTION (ADJUVANT EFFECT)
INCREASED LYMPHOKINE SYNTHESIS (IL-2, -3, -4, -5, -6, -7, -10 AND
-12)
INCREASED IL-2 (β) RECEPTOR
DEVELOPMENT OF TYPE 2 HUMAN T-CELL CLONES
INHIBITION OF TOLERANCE TO PROTEIN ANTIGENS
ENHANCEMENT OF SPLEEN CELL MITOGENIC RESPONSE TO LPS

EFFECTS OF T1-R LIGAND III ON CULTURED CELLS OR TISSUES

```
INCREASED EXPRESSION OF ELAM-1, VCAM-1, ICAM-1
CYTOTOXICITY (APOPTOSIS) OF INSULIN-PRODUCING ISLET β CELLS
INHIBITION OF THYROGLOBULIN SYNTHESIS IN THYROCYTES
CARTILAGE BREAKDOWN, RELEASE OF CALCIUM FROM BONE
INCREASED RELEASE OF ARACHIDONIC ACID, PROSTANOIDS, AND
  EICOSANOIDS
INCREASED MUCUS PRODUCTION AND CHLORIDE FLUX IN INTESTINAL
  CELLS
ENHANCEMENT IN CHLORIDE FLUX (GABAA RECEPTOR) IN BRAIN
  SYNAPTOSOMES
PROLIFERATION OF FIBROBLASTS, SMOOTH MUSCLE CELLS, MESSANGIAL
  CELLS
GROWTH INHIBITION OF HAIR FOLLICLES
INCREASED CORTICOSTERONE SYNTHESIS BY ADRENALS
INCREASED HIV OR OTHER RETROVIRAL EXPRESSION
```

ASSAYS USED: PANCREATIC ENDOCRINE TISSUE (MANDRUP-POULSEN, T., ET AL., CYTOKINE 5:185 (1993)), THYROID GLAND (RASMUSSEN, A. K., AUTOIMMUNITY 16:141 (1993)), HYPOTHALAMIC-PITUITARY-ADRENAL AXIS (FANTUZZI, G., & GHEZZI, P., MEDIATOR INFLAMM. 2:263 (1993); RIVIER, C., ANN. NY ACAD. Sci. 697:97 (1993); RIVIER, C., & RIVEST, S., CIBA. FOUND. SYMP. 172:204 (1993)), FEVER (COCEANI, F., “FEVER: BASIC MECHANISMS AND MANAGEMENT”, NEW YORK, N.Y., RAVEN (1991) P. 59), BONE METABOLISM (TATAKIS, D. N., J. PERIDONTOL 64:416 (1993)), DESTRUCTION OF CARTILAGE IN THE PATHOGENESIS OF RHEUMATOID ARTHRITIS (AREND, W. P., & DAYER, J. M., ARTHRITIS RHEUM 33:305 (1990); KRANE, S. M., ET AL., ANN. NY ACAD. Sci. 580:340 (1990)), UTERINE IMPLANTATION (LEWIS, M. P., ET AL., PLACENTA 15:13 (1994)), AND LOSS OF LEAN BODY MASS (ROUBENOFF, R., ET AL., J. CLIN. INVEST. 93:2379 (1994).

Gene Mapping

The nucleic acid molecules of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

In certain preferred embodiments in this regard, the cDNA herein disclosed is used to clone genomic DNA of a T1-R ligand III protein gene. This can be accomplished using a variety of well known techniques and libraries, which generally are available commercially. The genomic DNA then is used for in situ chromosome mapping using well known techniques for this purpose.

In addition, in some cases, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Fluorescence in situ hybridization (“FISH”) of a CDNA clone to a metaphase chromosomal spread can be used to provide a precise chromosomal location in one step. This technique can be used with probes from the cDNA as short as 50 or 60 bp. For a review of this technique, see Verma et al., Human Chromosomes: A Manual Of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance In Man, available on-line through Johns Hopkins University, Welch Medical Library. The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

Having generally described the invention, the same will be more readily understood by reference to the following examples, which are provided by way of illustration and are not intended as limiting.

### EXAMPLES

### Example 1

### Expression and Purification of T1-R Ligand III in E. coli

The bacterial expression vector pQE60 is used for bacterial expression in this example (QIAGEN, Inc., 9259 Eton Avenue, Chatsworth, Calif., 91311). pQE60 encodes ampicillin antibiotic resistance (“Ampr”) and contains a bacterial origin of replication (“ori”), an IPTG inducible promoter, a ribosome binding site (“RBS”), six codons encoding histidine residues that allow affinity purification using nickel-nitrilo-tri-acetic acid (“Ni-NTA”) affinity resin sold by QIAGEN, Inc., supra, and suitable single restriction enzyme cleavage sites. These elements are arranged such that a DNA fragment encoding a polypeptide may be inserted in such as way as to produce that polypeptide with the six His residues (i.e., a “6× His tag”) covalently linked to the carboxyl terminus of that polypeptide. However, in this example, the polypeptide coding sequence is inserted such that translation of the six His codons is prevented and, therefore, the polypeptide is produced with no 6× His tag.

The DNA sequence encoding the desired portion of the T1-R ligand III protein comprising the mature form of the T1-R ligand III amino acid sequence is amplified from the deposited cDNA clone using PCR oligonucleotide primers which anneal to the amino terminal sequences of the desired portion of the T1-R ligand III protein and to sequences in the deposited construct 3′ to the cDNA coding sequence. Additional nucleotides containing restriction sites to facilitate cloning in the pQE60 vector are added to the 5′ and 3′ sequences, respectively. For cloning the mature form of the T1-R ligand III protein, the 5′ primer has the sequence 5′ CGCCCATGGAGATCACCTTCGAGCTTC 3′ (SEQ ID NO:10) containing the underlined Nco I restriction site followed by 22 nucleotides of the amino terminal coding sequence of the mature T1-R ligand III sequence in SEQ ID NO:2. One of ordinary skill in the art would appreciate, of course, that the point in the protein coding sequence where the 5′ primer begins may be varied to amplify a desired portion of the complete protein shorter or longer than the mature form. The 3′ primer has the sequence 5′ CGCAAGCTTGAAATGCCTACATGTAAG 3′ (SEQ ID NO: 11) containing the underlined Hind III restriction site followed by 18 nucleotides complementary to the 3′ end of the coding sequence in the T1-R ligand III DNA sequence in FIG. 1. Alternatively, the 3′ primer has the sequence 5′ CGCAAGCTTCAGCAATATTACAGTGGC 3′ (SEQ ID NO: 12) containing the underlined Hind III restriction site followed by 18 nucleotides complementary to the 3′ end of the coding sequence in the T1-R ligand III DNA sequence in FIG. 2.

The amplified T1-R ligand III DNA fragments and the vector pQE60 are digested with Nco I and Hind III and the digested DNAs are then ligated together. Insertion of the T1-R ligand III DNA into the restricted pQE60 vector places the T1-R ligand III protein coding region including its associated stop codon downstream from the IPTG-inducible promoter and in-frame with an initiating AUG. The associated stop codon prevents translation of the six histidine codons downstream of the insertion point.

The ligation mixture is transformed into competent E. coli cells using standard procedures such as those described in Sambrook et al., Molecular Cloning: a Laboratory Manual, 2nd Ed.; Cold Spring Harbor Laboratory Press, Cold Spring Harbor, N.Y. (1989). E. coli strain M15/rep4, containing multiple copies of the plasmid pREP4, which expresses the lac repressor and confers kanamycin resistance (“Kanr”), is used in carrying out the illustrative example described herein. This strain, which is only one of many that are suitable for expressing T1-R ligand III protein, is available commercially from QIAGEN, Inc., supra. Transformants are identified by their ability to grow on LB plates in the presence of ampicillin and kanamycin. Plasmid DNA is isolated from resistant colonies and the identity of the cloned DNA confirmed by restriction analysis, PCR and DNA sequencing.

Clones containing the desired constructs are grown overnight (“O/N”) in liquid culture in LB media supplemented with both ampicillin (100 μg/ml) and kanamycin (25 μg/ml). The O/N culture is used to inoculate a large culture, at a dilution of approximately 1:25 to 1:250. The cells are grown to an optical density at 600 nm (“OD600”) of between 0.4 and 0.6. isopropyl-b-D-thiogalactopyranoside (“IPTG”) is then added to a final concentration of 1 mM to induce transcription from the lac repressor sensitive promoter, by inactivating the lacI repressor. Cells subsequently are incubated further for 3 to 4 hours. Cells then are harvested by centrifugation.

To purify the T1-R ligand III polypeptide, the cells are then stirred for 3-4 hours at 4° C. in 6M guanidine-HCl, pH 8. The cell debris is removed by centrifugation, and the supernatant containing the T1-R ligand III is dialyzed against 50 mM Na-acetate buffer pH 6, supplemented with 200 mM NaCl. Alternatively, the protein can be successfully refolded by dialyzing it against 500 mM NaCl, 20% glycerol, 25 mM Tris/HCl pH 7.4, containing protease inhibitors. After renaturation the protein can be purified by ion exchange, hydrophobic interaction and size exclusion chromatography. Alternatively, an affinity chromatography step such as an antibody column can be used to obtain pure T1-R ligand III protein. The purified protein is stored at 4° C. or frozen at −80° C.

### Example 2

### Cloning and Expression of T1-R Ligand III Protein in a Baculovirus Expression System

In this illustrative example, the plasmid shuttle vector pA2 is used to insert the cloned DNA encoding complete protein, including its naturally associated secretory signal (leader) sequence, into a baculovirus to express the mature T1-R ligand III protein, using standard methods as described in Summers and colleagues (“A Manual of Methods for Baculovirus Vectors and Insect Cell Culture Procedures”, Texas Agricultural Experimental Station Bulletin No. 1555 (1987)). This expression vector contains the strong polyhedrin promoter of the Autographa californica nuclear polyhedrosis virus (AcMNPV) followed by convenient restriction sites such as Bam HI, Xba I and Asp 718. The polyadenylation site of the simian virus 40 (“SV40”) is used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

The cDNA sequence encoding the full length T1-R ligand III protein in the deposited clone, including the AUG initiation codon and the naturally associated leader sequence shown in SEQ ID NO:2 or in SEQ ID NO:4, is amplified using PCR oligonucleotide primers corresponding to the 5′ and 3′ sequences of the gene. The 5′ primer has the sequence 5′ CGCGGATCCGCCATCATGGGCGGCCGTCGCGGGCGTTG 3′ (SEQ ID NO:13) containing the underlined Bam HI restriction enzyme site, an efficient signal for initiation of translation in eukaryotic cells, as described by Kozak, M., J. Mol. Biol. 196:947-950 (1987), followed by 29 of nucleotides of the sequence of the complete T1-R ligand III protein shown in FIG. 1 or FIG. 2, beginning with the AUG initiation codon. The 3′ primer has the sequence 5′ CGCGGTACCGAAATGCCTACATGTAAG 3′ (SEQ ID NO:14) containing the underlined Asp 718 restriction site followed by 18 nucleotides complementary to the 3′ noncoding sequence in FIG. 1. Alternatively, the 3′ primer has the sequence 5′ CGCGGTACCCAGCAATATTACAGTGGC 3′ (SEQ ID NO:15) containing the underlined Asp 718 restriction site followed by 18 nucleotides complementary to the 3′ noncoding sequence in FIG. 2.

Many other baculovirus vectors could be used in place of the vector above, such as pAc373, pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

The amplified fragment is isolated from a 1% agarose gel using a commercially available kit (“Geneclean,” BIO 101 Inc., La Jolla, Calif.). The fragment then is digested with Bam HI and Asp 718 and again is purified on a 1% agarose gel. This fragment is designated herein F1.

The plasmid is digested with the restriction enzymes Bam HI and Asp 718 and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.). This vector DNA is designated herein “V1”.

Fragment F1 and the dephosphorylated plasmid V1 are ligated together with T4 DNA ligase. E. coli HB101 or other suitable E. coli hosts such as XL-1 Blue (Statagene Cloning Systems, La Jolla, Calif.) cells are transformed with the ligation mixture and spread on culture plates. Bacteria are identified that contain the plasmid with the human T1-R ligand III gene by digesting DNA from individual colonies using Bam HI and Asp 718 and then analyzing the digestion product by gel electrophoresis. The sequence of the cloned fragment is confirmed by DNA sequencing. This plasmid is designated herein pA2T1-R ligand III.

Five μg of the plasmid pA2T1-R ligand III is co-transfected with 1.0 μg of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84: 7413-7417 (1987). One μg of BaculoGold™ virus DNA and 5 μg of the plasmid pA2T1-R ligand III are mixed in a sterile well of a microtiter plate containing 50 μl of serum-free Grace's medium (Life Technologies Inc., Gaithersburg, Md.). Afterwards, 10 μl Lipofectin plus 90 μl Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27° C. The transfection solution is then removed from the plate and 1 ml of Grace's insect medium supplemented with 10% fetal calf serum is added. Cultivation is then continued at 27° C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10). After appropriate incubation, blue stained plaques are picked with the tip of a micropipettor (e.g., Eppendorf). The agar containing the recombinant viruses is then resuspended in a microcentrifuge tube containing 200 μl of Grace's medium and the suspension containing the recombinant baculovirus is used to infect Sf9 cells seeded in 35 mm dishes. Four days later the supernatants of these culture dishes are harvested and then they are stored at 4° C. The recombinant virus is called V-T1-R ligand III.

To verify the expression of the T1-R ligand III gene Sf9 cells are grown in Grace's medium supplemented with 10% heat-inactivated FBS. The cells are infected with the recombinant baculovirus V-T1-R ligand III at a multiplicity of infection (“MOI”) of about 2. If radiolabeled proteins are desired, 6 hours later the medium is removed and is replaced with SF900 II medium minus methionine and cysteine (available from Life Technologies Inc., Rockville, MD). After 42 hours, 5 μCi of ³⁵S-methionine and 5 μCi ³⁵S-cysteine (available from Amersham) are added. The cells are further incubated for 16 hours and then are harvested by centrifugation. The proteins in the supernatant as well as the intracellular proteins are analyzed by SDS-PAGE followed by autoradiography (if radiolabeled).

Microsequencing of the amino acid sequence of the amino terminus of purified protein may be used to determine the amino terminal sequence of the mature form of the T1-R ligand III protein and thus the cleavage point and length of the naturally associated secretory signal peptide.

### Example 3

### Cloning and Expression of T1-R Ligand III in Mammalian Cells

A typical mammalian expression vector contains the promoter element, which mediates the initiation of transcription of mRNA, the protein coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Mammalian host cells that could be used include, human Hela, 293, H9 and Jurkat cells, mouse NIH3T3 and C127 cells, Cos 1, Cos 7 and CVI, quail QC 1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, hygromycin allows the identification and isolation of the transfected cells.

The transfected gene can also be amplified to express large amounts of the encoded protein. The DHFR (dihydrofolate reductase) marker is useful to develop cell lines that carry several hundred or even several thousand copies of the gene of interest. Another useful selection marker is the enzyme glutarnine synthase (GS; Murphy et al., Biochem J. 227:277-279 (1991); Bebbington et al., Bio/Technology 10:169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

The expression vectors pC1 and pC4 contain the strong promoter (LTR) of the Rous Sarcoma Virus (Cullen et al., Molecular and Cellular Biology, 438-447 (March, 1985)) plus a fragment of the CMV-enhancer (Boshart et al., Cell 41:521-530 (1985)). Multiple cloning sites, e.g., with the restriction enzyme cleavage sites Bam HI, Xba I and Asp 718, facilitate the cloning of the gene of interest. The vectors contain in addition the 3′ intron, the polyadenylation and termination signal of the rat preproinsulin gene.

### Example 3(a)

### Cloning and Expression in COS Cells

The expression plasmid, pT1-R ligand IIIHA, is made by cloning a portion of the cDNA encoding the mature form of the T1-R ligand III protein into the expression vector pcDNAI/Amp or pcDNAIII (which can be obtained from Invitrogen, Inc.).

The expression vector pcDNAI/amp contains: (1) an E. coli origin of replication effective for propagation in E. coli and other prokaryotic cells; (2) an ampicillin resistance gene for selection of plasmid-containing prokaryotic cells; (3) an SV40 origin of replication for propagation in eukaryotic cells; (4) a CMV promoter, a polylinker, an SV40 intron; (5) several codons encoding a hemagglutinin fragment (i.e., an “HA” tag to facilitate purification) followed by a termination codon and polyadenylation signal arranged so that a cDNA can be conveniently placed under expression control of the CMV promoter and operably linked to the SV40 intron and the polyadenylation signal by means of restriction sites in the polylinker. The HA tag corresponds to an epitope derived from the influenza hemagglutinin protein described by Wilson and coworkers (Cell 37: 767 (1984)). The fusion of the HA tag to the target protein allows easy detection and recovery of the recombinant protein with an antibody that recognizes the HA epitope. pcDNAIII contains, in addition, the selectable neomycin marker. A DNA fragment encoding the complete T1-R ligand III polypeptide is cloned into the polylinker region of the vector so that recombinant protein expression is directed by the CMV promoter. The plasmid construction strategy is as follows. The T1-R ligand III cDNA of the deposited clone is amplified using primers that contain convenient restriction sites, much as described above for construction of vectors for expression of T1-R ligand III in E. coli. Suitable primers include the following, which are used in this example. The 5′ primer, containing the underlined Bam HI site, a Kozak sequence, an AUG start codon, and 23 nucleotides of the 5′ coding region of the complete T1-R ligand III polypeptide, has the following sequence: 5′ CGCGGATCCGCCATCAT GGGCGGCCGTCGCGGGCGTTG 3′ (SEQ ID NO:13). The 3′ primer has the sequence 5′ CGCGGTACCGAAATGCCTACATGTAAG 3′ (SEQ ID NO:14) containing the underlined Asp 718 restriction site followed by 18 nucleotides complementary to the 3′ noncoding sequence in FIG. 1. Alternatively, the 3′ primer has the sequence 5′ CGCGGTACCCAGCAATA TTACAGTGGC 3′ (SEQ ID NO:15) containing the underlined Asp 718 restriction site followed by 18 nucleotides complementary to the 3′ noncoding sequence in FIG. 2.

The PCR amplified DNA fragment and the vector, pcDNAI/Amp, are digested with Bam HI and Asp 718 and then ligated. The ligation mixture is transformed into E. coli strain SURE (available from Stratagene Cloning Systems, 11099 North Torrey Pines Road, La Jolla, Calif. 92037), and the transformed culture is plated on ampicillin media plates which then are incubated to allow growth of ampicillin resistant colonies. Plasmid DNA is isolated from resistant colonies and examined by restriction analysis or other means for the presence of the fragment encoding the complete T1-R ligand III polypeptide For expression of recombinant T1-R ligand III, COS cells are transfected with an expression vector, as described above, using DEAE-DEXTRAN, as described, for instance, by Sambrook and coworkers (Molecular Cloning: a Laboratory Manual, Cold Spring Laboratory Press, Cold Spring Harbor, N.Y. (1989)). Cells are incubated under conditions for expression of T1-R ligand III by the vector.

Expression of the T1-R ligand III-HA fusion protein is detected by radiolabeling and immunoprecipitation, using methods described by, for example Harlow and colleagues (Antibodies: A Laboratory Manual, 2nd Ed.; Cold Spring Harbor Laboratory Press, Cold Spring Harbor, N.Y. (1988)). To this end, two days after transfection, the cells are labeled by incubation in media containing ³⁵S-cysteine for 8 hours. The cells and the media are collected, and the cells are washed and the lysed with detergent-containing RIPA buffer: 150 mM NaCl, 1% NP-40, 0.1% SDS, 1% NP-40, 0.5% DOC, 50 mM TRIS, pH 7.5, as described by Wilson et al. cited above. Proteins are precipitated from the cell lysate and from the culture media using an HA-specific monoclonal antibody. The precipitated proteins then are analyzed by SDS-PAGE and autoradiography. An expression product of the expected size is seen in the cell lysate, which is not seen in negative controls.

### Example 3(b)

### Cloning and Expression in CHO Cells

The vector pC4 is used for the expression of T1-R ligand III polypeptide. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (alpha minus MEM, Life Technologies) supplemented with the chemotherapeutic agent methotrexate. The amplification of the DHFR genes in cells resistant to methotrexate (MTX) has been well documented (see, e.g., Alt, F. W., Kellems, R. M., Bertino, J. R., and Schimke, R. T., 1978, J. Biol. Chem. 253:1357-1370, Hamlin, J. L. and Ma, C. 1990, Biochem. et Biophys. Acta, 1097:107-143, Page, M. J. and Sydenham, M. A. 1991, Biotechnology 9:64-68). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach may be used to develop cell lines carrying more than 1,000 copies of the amplified gene(s). Subsequently, when the methotrexate is withdrawn, cell lines are obtained which contain the amplified gene integrated into one or more chromosome(s) of the host cell.

Plasmid pC4 contains for expressing the gene of interest the strong promoter of the long terminal repeat (LTR) of the Rouse Sarcoma Virus (Cullen, et al., Mol. Cell. Biol., March 1985:438-447) plus a fragment isolated from the enhancer of the immediate early gene of human cytomegalovirus (CMV) (Boshart et al., Cell 41:521-530 (1985)). Downstream of the promoter are the following single restriction enzyme cleavage sites that allow the integration of the genes: BamHI, Xba I, and Asp718. Behind these cloning sites the plasmid contains the 3′ intron and polyadenylation site of the rat preproinsulin gene. Other high efficiency promoters can also be used for the expression, e.g., the human B-actin promoter, the SV40 early or late promoters or the long terminal repeats from other retroviruses, e.g., HIV and HTLVI. Clontech's Tet-Off and Tet-On gene expression systems and similar systems can be used to express the T1-R ligand III polypeptide in a regulated way in mammalian cells (Gossen, M., & Bujard, H., Proc. Natl. Acad. Sci. USA 89:5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with the restriction enzymes Bam HI and Asp 718 and then dephosphorylated using calf intestinal phosphates by procedures known in the art. The vector is then isolated from a 1% agarose gel. The DNA sequence encoding the complete T1-R ligand III polypeptide is amplified using PCR oligonucleotide primers corresponding to the 5′ and 3′ sequences of the desired portion of the gene. The 5′ primer, containing the underlined Bam HI site, a Kozak sequence, an AUG start codon, and 23 nucleotides of the 5′ coding region of the complete T1-R ligand III polypeptide, has the following sequence: 5′ CGCGGATCCGCCATCATGG GCGGCCGTCGCGGGCGTTG 3′ (SEQ ID NO:13). The 3′ primer has the sequence 5′ CGCGGTACCGAAATGCCTACATGTAAG 3′ (SEQ ID NO:14) containing the underlined Asp 718 restriction site followed by 18 nucleotides complementary to the 3′ noncoding sequence in FIG. 1. Alternatively, the 3′ primer has the sequence 5′ CGCGGTACCCAGCAATA TTACAGTGGC 3′ (SEQ ID NO:15) containing the underlined Asp 718 restriction site followed by 18 nucleotides complementary to the 3′ noncoding sequence in FIG. 2.

The amplified fragment is digested with the endonucleases Bam HI and Asp 718 and then purified again on a 1% agarose gel. The isolated fragment and the dephosphorylated vector are then ligated with T4 DNA ligase. E. coli HB 101 or XL-1 Blue cells are then transformed and bacteria are identified that contain the fragment inserted into plasmid pC4 using, for instance, restriction enzyme analysis.

Chinese hamster ovary cells lacking an active DHFR gene are used for transfection. Five μg of the expression plasmid pC4 is cotransfected with 0.5 μg of the plasmid pSVneo using lipofectin (Felgner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of metothrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 μM, 2 μM, 5 μM, 10 mM, 20 mM). The same procedure is repeated until clones are obtained which grow at a concentration of 100-200 μM. Expression of the desired gene product is analyzed, for instance, by SDS-PAGE and Western blot or by reversed phase HPLC analysis.

### Example 4

### Tissue Distribution of T1-R Ligand III mRNA Expression

Northern blot analysis is carried out to examine T1-R ligand III gene expression in human tissues, using methods described by, among others, Sambrook et al., cited above. A cDNA probe containing the entire nucleotide sequence of the T1-R ligand III protein (SEQ ID NO:1) is labeled with ³²P using the rediprime™ DNA labeling system (Amersham Life Science), according to manufacturer's instructions. After labeling, the probe is purified using a CHROMA SPIN-100™ column (Clontech Laboratories, Inc.), according to manufacturer's protocol number PT1200-1. The purified labeled probe is then used to examine various human tissues for T1-R ligand III mRNA.

Multiple Tissue Northern (MTN) blots containing various human tissues (H) or human immune system tissues (IM) are obtained from Clontech and are examined with the labeled probe using ExpressHyb™ hybridization solution (Clontech) according to manufacturer's protocol number PT1190-1. Following hybridization and washing, the blots are mounted and exposed to film at −70° C. overnight, and films developed according to standard procedures.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of all publications (including patents, patent applications, journal articles, laboratory manuals, books, or other documents) cited herein are hereby incorporated by reference.

What is claimed is:

1. An isolated nucleic acid molecule comprising a polynucleotide having a nucleotide sequence at least 95% identical to a sequence selected from the group consisting of:
(a) a nucleotide sequence encoding the T1-R ligand III polypeptide having the amino acid sequence at positions −24 to 139 of SEQ ID NO:2 or the complete amino acid sequence encoded by the cDNA clone contained in ATCC Deposit No. 97859
(b) a nucleotide sequence encoding the T1-R ligand III polypeptide having the amino acid sequence at positions −24 to 191 of SEQ ID NO:4 or the complete amino acid sequence encoded by the cDNA clone contained in ATCC Deposit No. 97858
(c) a nucleotide sequence encoding the T1-R ligand III polypeptide having the amino acid sequence at positions −23 to 139 of SEQ ID NO:2 or the complete amino acid sequence excepting the N-terminal methionine encoded by the cDNA clone contained in ATCC Deposit No. 97859
(d) a nucleotide sequence encoding the T1-R ligand III polypeptide having the amino acid sequence at positions −23 to 191 of SEQ ID NO:4 or the complete amino acid sequence excepting the N-terminal methionine encoded by the cDNA clone contained in ATCC Deposit No. 97858
(e) a nucleotide sequence encoding the mature form of the T1-R ligand III polypeptide having the amino acid sequence at positions 1 to 139 in SEQ ID NO:2, or as encoded by the cDNA clone contained in the ATCC Deposit No. 97859;
(f) a nucleotide sequence encoding the mature form of the T1-R ligand III polypeptide having the amino acid sequence at positions 1 to 191 in SEQ ID NO:4, or as encoded by the cDNA clone contained in the ATCC Deposit No. 97858;
(g) a nucleotide sequence complementary to any of the nucleotide sequences in (a), (b), (c), (d), (e) or (f) above.

2. The nucleic acid molecule of claim 1 wherein said polynucleotide has the complete nucleotide sequence in FIG. 1 (SEQ ID NO: 1) or FIG. 2 (SEQ ID NO:3)

3. The nucleic acid molecule of claim 1 wherein said polynucleotide has the nucleotide sequence in FIG. 1 (SEQ ID NO: 1) or FIG. 2 (SEQ ID NO:3) encoding the T1-R ligand III polypeptide having the amino acid sequence in positions −23 to 139 of SEQ ID NO:2 or having the amino acid sequence in positions −23 to 191 of SEQ ID NO:4, respectively.

4. The nucleic acid molecule of claim 1 wherein said polynucleotide has the nucleotide sequence in FIG. 1 (SEQ ID NO:1) or FIG. 2 (SEQ ID NO:3) encoding the mature T1-R ligand III polypeptide having the amino acid sequence from about 1 to about 139 in SEQ ID NO:2 or having the amino acid sequence from about 1 to about 191 in SEQ ID NO:4, respectively.

5. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n-139 of SEQ ID NO:2, where n is an integer in the range of −23-15;
(b) a nucleotide sequence encoding a polypeptide comprising the amino acid sequence of residues −23-m of SEQ ID NO:2, where m is an integer in the range of 129 to 138;
(c) a nucleotide sequence encoding a polypeptide having the amino acid sequence consisting of residues n-m of SEQ ID NO:2, where n and m are integers as defined respectively in (a) and (b) above; and
(d) a nucleotide sequence encoding a polypeptide consisting of a portion of the complete T1-R ligand III amino acid sequence encoded by the cDNA clone contained in ATCC Deposit No. 97859 wherein said portion excludes from 1 to about 14 amino acids from the amino terminus of said complete amino acid sequence encoded by the cDNA clone contained in ATCC Deposit No. 97859;
(e) a nucleotide sequence encoding a polypeptide consisting of a portion of the complete T1-R ligand III amino acid sequence encoded by the cDNA clone contained in ATCC Deposit No. 97859 wherein said portion excludes from 1 to about 10 amino acids from the carboxy terminus of said complete amino acid sequence encoded by the cDNA clone contained in ATCC Deposit No. 97859; and
(f) a nucleotide sequence encoding a polypeptide consisting of a portion of the complete T1-R ligand III amino acid sequence encoded by the cDNA clone contained in ATCC Deposit No. 97859 wherein said portion include a combination of any of the amino terminal and carboxy terminal deletions in (d) and (e), above.

6. An isolated nucleic acid molecule comprising a polynucleotide having a nucleotide sequence at least 95% identical to a sequence selected from the group consisting of:
(a) a nucleotide sequence encoding a polypeptide comprising the amino acid sequence of residues n-139 of SEQ ID NO:4, where n is an integer in the range of −23-15;
(b) a nucleotide sequence encoding a polypeptide comprising the amino acid sequence of residues −23-m of SEQ ID NO:4, where m is an integer in the range of 181 to 190;
(c) a nucleotide sequence encoding a polypeptide having the amino acid sequence consisting of residues n-m of SEQ ID NO:4, where n and m are integers as defined respectively in (a) and (b) above; and
(d) a nucleotide sequence encoding a polypeptide consisting of a portion of the complete T1-R ligand III amino acid sequence encoded by the cDNA clone contained in ATCC Deposit No. 97858 wherein said portion excludes from 1 to about 14 amino acids from the amino terminus of said complete amino acid sequence encoded by the cDNA clone contained in ATCC Deposit No. 97858;
(e) a nucleotide sequence encoding a polypeptide consisting of a portion of the complete T1-R ligand III amino acid sequence encoded by the cDNA clone contained in ATCC Deposit No. 97858 wherein said portion excludes from 1 to about 10 amino acids from the carboxy terminus of said complete amino acid sequence encoded by the cDNA clone contained in ATCC Deposit No. 97858; and
(f) a nucleotide sequence encoding a polypeptide consisting of a portion of the complete T1-R ligand III amino acid sequence encoded by the cDNA clone contained in ATCC Deposit No. 97858 wherein said portion include a combination of any of the amino terminal and carboxy terminal deletions in (d) and (e), above.

7. The nucleic acid molecule of claim 1 wherein said polynucleotide has the complete nucleotide sequence of the cDNA clone contained in ATCC Deposit No. 97859 or in ATCC Deposit No. 97858.

8. The nucleic acid molecule of claim 1 wherein said polynucleotide has the nucleotide sequence encoding the T1-R ligand III polypeptide having the complete amino acid sequence excepting the N-terminal methionine encoded by the cDNA clone contained in ATCC Deposit No. 97859 or in ATCC Deposit No. 97858.

9. The nucleic acid molecule of claim 1 wherein said polynucleotide has the nucleotide sequence encoding the mature T1-R ligand III polypeptide having the amino acid sequence encoded by the cDNA clone contained in ATCC Deposit No. 97859 or in ATCC Deposit No. 97858.

10. An isolated nucleic acid molecule comprising a polynucleotide which hybridizes under stringent hybridization conditions to a polynucleotide having a nucleotide sequence identical to a nucleotide sequence in (a), (b), (c), (d) or (e) of claim 1 wherein said polynucleotide which hybridizes does not hybridize under stringent hybridization conditions to a polynucleotide having a nucleotide sequence consisting of only A residues or of only T residues.

11. An isolated nucleic acid molecule comprising a polynucleotide which encodes the amino acid sequence of an epitope-bearing portion of a T1-R ligand III polypeptide having an amino acid sequence in (a), (b), (c) or (d) of claim 1.

12. The isolated nucleic acid molecule of claim 11, which encodes an epitope-bearing portion of a T1-R ligand III polypeptide wherein the amino acid sequence of said portion is selected from the group of sequences in SEQ ID NO:2 consisting of: about 1 to about 10, about 30 to about 40, about 45 to about 57, about 89 to about 100, about 120 to about 132, and about 150 to about 162.

13. The isolated nucleic acid molecule of claim 11, which encodes an epitope-bearing portion of a T1-R ligand III polypeptide wherein the amino acid sequence of said portion is selected from the group of sequences in SEQ ID NO:4 consisting of: about 1-10, about 30 to about 40, about 45 to about 57, about 89 to about 100, about 120 to about 132, about 155 to about 165, and about 201 to about 212.

14. A method for making a recombinant vector comprising inserting an isolated nucleic acid molecule of claim 1 into a vector.

15. A recombinant vector produced by the method of claim 14.

16. A method of making a recombinant host cell comprising introducing the recombinant vector of claim 15 into a host cell.

17. A recombinant host cell produced by the method of claim 16.

18. A recombinant method for producing a T1-R ligand III polypeptide, comprising culturing the recombinant host cell of claim 17 under conditions such that said polypeptide is expressed and recovering said polypeptide.

19. An isolated T1-R ligand III polypeptide comprising an amino acid sequence at least 95% identical to a sequence selected from the group consisting of:
(a) the amino acid sequence positions −23 to 139 of SEQ ID NO:2 or amino acid sequence positions −23 to 191 of SEQ ID NO:4 or the complete T1-R ligand III amino acid sequence excepting the N-terminal methionine encoded by the cDNA clone contained in ATCC Deposit No. 97859 or in ATCC Deposit No. 97858;
(b) the amino acid sequence of the mature T1-R ligand III polypeptide having the amino acid sequence at positions 1 to 139 in SEQ ID NO:2 or amino acid sequence at positions 1 to 191 in SEQ ID NO:4, or as encoded by the cDNA clone contained in the ATCC Deposit No. 97859 or in ATCC Deposit No. 97858;

20. An isolated polypeptide comprising an epitope-bearing portion of the T1-R ligand III protein, wherein said portion is selected from the group consisting of: a polypeptide comprising amino acid residues from about 1 to about 10, about 30 to about 40, about 45 to about 57, about 89 to about 100, about 120 to about 132, and about 150 to about 162 of SEQ ID NO:2

21. An isolated polypeptide comprising an epitope-bearing portion of the T1-R ligand III protein, wherein said portion is selected from the group consisting of: a polypeptide comprising amino acid residues from about 1-10, about 30 to about 40, about 45 to about 57, about 89 to about 100, about 120 to about 132, about 155 to about 165, and about 201 to about 212.

22. An isolated antibody that binds specifically to a T1-R ligand III polypeptide of claim 19.

23. An isolated nucleic acid molecule comprising a polynucleotide having a sequence at least 95% identical to a sequence selected from the group consisting of:
(a) the nucleotide sequence of clone HETDW91R (SEQ ID NO:6);
(b) the nucleotide sequence of clone HSRDN17R (SEQ ID NO:7);
(c) the nucleotide sequence of a portion of the sequence shown in FIG. 1 (SEQ ID NO:1) wherein said portion comprises at least 50 contiguous nucleotides from a nucleotide sequence selected from the group consisting of about 1 to about 100, about 450 to about 615, and about 690 to about 1836; and
(d) a nucleotide sequence complementary to any of the nucleotide sequences in (a), (b) or (c) above.

24. An isolated nucleic acid molecule comprising a polynucleotide having a sequence at least 95% identical to a sequence selected from the group consisting of:
(a) the nucleotide sequence of clone HETDW91R (SEQ ID NO:6);
(b) the nucleotide sequence of clone HSRDN17R (SEQ ID NO:7);
(c) the nucleotide sequence of clone HASAA3 IR (SEQ ID NO:8);
(d) the nucleotide sequence of clone HPFCQ85R (SEQ ID NO:9); and
(e) the nucleotide sequence of a portion of the sequence shown in FIG. 2 (SEQ ID NO:3) wherein said portion comprises at least 50 contiguous nucleotides from nucleotide about 1 to about 100, about 1000 to about 1390, and about 1430 to about 1513; and
(f) a nucleotide sequence complementary to any of the nucleotide sequences in (a), (b), (c), (d) or (e) above.


SEQUENCE LISTING (16 sequences)

SEQ ID NO: 1 - DNA, 1836 bp, Homo sapiens
  mat_peptide (103)..(519)
  sig_peptide (31)..(102)
  CDS (31)..(522)
gcacgagccgcggccggggtccgcgcagcgatgggcggccgtcgcgggcgttgggggtgcaggctgctcgcactgctgctactggtgcctggacccggcggcgcctctgagatcaccttcgagcttcctgacaacgccaagcagtgcttctacgaggacatcgctcagggcaccaagtgcaccctggagttccaggtgattactggtggtcactatgatgtagattgtcgattagaagatcctgatggtaaagtgttatacaaagagatgaagaaacagtatgatagttttaccttcacagcctccaaaaatgggacatacaaattttgcttcagcaatgaattttctactttcacacataaaactgtatattttgattttcaagttggagaagacccacctttgtttcctagtgagaaccgagtcagtgctcttacccaggtaaataaaaaaatcagcaatataatgttggtatatttaaaaggaggaaaaaagcataatttaatatcatgcactaactgatgagcatggaattttgagactgacagtctttaaaatctatcttaattcttacatgtaggcatttcacagagaacttcagacttttgtagtgaaaaataatgttgaaagattttatgaacttttaattgtaaagagaaagaagcattaaaataaggaggaagatcactaaggtagaaacatcagtggtaaaagaaggttgggaaggctgagcagtgttccaatcttctgaagcattttctttaatccctgaaggaatgttgctgtcatactcaggaggctggtgtaactggaagcaaaatgatgcctaaattagtagataaataataccatatcatagcctttataccattacttagtgttactcaagctgctgtaacagtaacttgtgatgtaactttaggtgaaatattgaagattaatggtaagtcatctcacaaaaatagttacaaaagctaaattcagatttattttggagacatagagaaagcgtaaaaaatttaaacccagtgaacaactattgcttaggtgatgaggactgttttatttctagttcaaatacagcaaaatccaaaatgcttcaaaatccaaaacttttttttagctccaacctgatgacacaagtggaagattgttacatctgatctcatgacacatcattgaaaatatcatatacagttgccttaaggctgcatgtataaggcatatatgaaacataaatgaatttcatgtctagacctgggttccatcacaaagatatctcattatgtatatgtagacattccaaaatcagaaaaaatctgaaattcaaagcacttctggtcccaaacattttggataagggatactcaacctgtatgggcttagatcgtatctaaaagttactcaattaggcatgaaaaagaattatttaccacttattcgttaacatcttaaactggtattttagtgttcccagggttctatttagtctcttggccaaagtcatggtgaaagaaacagctttaagaatgtgtaaaagccttaattcataaacctgttgttagaattagcaagatatattcaaatactcttaattttagaaatgttctctttattttaaggtcttgtgtgaaggaccccttgctttgtaaaatgatatttctttggggccattaccttaacctgtatccattccttcatgaattttaacttgtataaggtggcttgttctaaaaacctaagtgggtaaatataagaaatgaatggtaataaagcagttagtatcataaaaaaaaaaaaaaaaaaaaaaaaaaaaaaaaaaaaaaaaaaaaaaaa

SEQ ID NO: 2 - protein, 163 aa, Homo sapiens
MGGRRGRWGCRLLALLLLVPGPGGASEITFELPDNAKQCFYEDIAQGTKCTLEFQVITGGHYDVDCRLEDPDGKVLYKEMKKQYDSFTFTASKNGTYKFCFSNEFSTFTHKTVYFDFQVGEDPPLFPSENRVSALTQVNKKISNIMLVYLKGGKKHNLISCTN

SEQ ID NO: 3 - DNA, 1585 bp, Homo sapiens
  CDS (28)..(672)
  mat_peptide (100)..(672)
  sig_peptide (28)..(99)
gcacagcccggccggggtccgcgcagcatgggcggccgtcgcgggccttgggggtgcaggctgctcgcactgctgctactggtgcctggacccggcggcgcctctgagatcaccttcgagcttcctgacaacgccaagcagtgcttctacgaggacatcgctcagggcaccaagtgcaccctggagttccaggtgattactggtggtcactatgatgtagattgtcgattagaagatcctgatggtaaagtgttatacaaagagatgaagaaacagtatgatagttttaccttcacagcctccaaaaatgggacatacaaattttgcttcagcaatgaattttctactttcacacataaaactgtatattttgattttcaagttggagaagacccacctttgtttcctagtgagaaccgagtcagtgctcttacccagatggaatctgcctgtgtttcaattcacgaagctctgaagtctgtcatcgattatcagactcatttccgtttaagagaagctcaaggccgaagccgagcagaggatctaaatacaagagtggcctattggtcagtaggagaagccctcattcttctggtggttagcatagggcaggtatttcttttgaaaagctttttctcagataaaagaaccaccacaactcgtgttggatcataactacgttttgagaattgatgcaccattgccactgtaatattgctgtcctctaattaattttaggtactgaagaacttaatattggcaacatttttaaatccttactcatacacttgttgggagggatgtacaatgcatattcccaaactgtggaaaggacacctttttttatttgtaaaggtggaaaaactttggaactcattttgggctattcatgttaaatattcaacaccaatgatctactctgttcgcagttgtttatatctactcttcgcacactaaactttggtattttgattccttttaaccatttaagactacttttcttataggtagttgatattttaaaaactttagatttaatgtctacatgtgttagggaggaagaaaattgccttttaattgttaataagaaaaccaaatgtgatgaactgtagcccaagccctattctgcactgttcagttttatggaggaaaaataaatctaccataggaatgttagttaatattgatatatcatggtaaaattgatttctcactagcttagaaaatgtcagacttttgtttttggggtttataatttaaaccagctatgctattttttcataaaggcatttgtagtacacagaaaaacagtagtttcagtagtgtaaaagagtttatacaggccttaaatatcagactttgtaacaggtagaaatattacagaaataatttaagacactacaatgggggcaaatgaaataggaaaatttttagtgagttacacgtactcattacattttcagtgcttttacaaggaaaaaaggtgatatgtttaattttaaaattttaattgggctagctcttgcccttatatgactttaatgtctgtgagtcattcccagcttaaattaacaattgtagtatt

SEQ ID NO: 4 - protein, 215 aa, Homo sapiens
MGGRRGPWGCRLLALLLLVPGPGGASEITFELPDNAKQCFYEDIAQGTKCTLEFQVITGGHYDVDCRLEDPDGKVLYKEMKKQYDSFTFTASKNGTYKFCFSNEFSTFTHKTVYFDFQVGEDPPLFPSENRVSALTQMESACVSIHEALKSVIDYQTHFRLREAQGRSRAEDLNTRVAYWSVGEALILLVVSIGQVFLLKSFFSDKRTTTTRVGS

SEQ ID NO: 5 - protein, 227 aa, Homo sapiens
MMAAGAALALALWLLMPPVEVGGAGPPPIQDGEFTFLLPAGRKQCFYQSAPANASLETEYQVIGGAGLDVDFTLESPQGVLLVSESRKADGVHTVEPTEAGDYKLCFDNSFSTISEKLVFFELIFDSLQDDEEVEGWAEAVEPEEMLDVKMEDIKESIETMRTRLERSIQMLTLLRAFEARDRNLQEGNLERVNFWSAVNVAVLLLVAVLQVCTLKRFFQDKRPVPT

SEQ ID NO: 6 - DNA, 312 bp, Homo sapiens
ggcaagccggccggggtccggcagctgggcggccgtcgcgggcctgtggggtgcaggctgctcgcactgctgcactggtgcctggacccggcggcgtcttctaaatcaccttcgaggcttcctggacaacgcaagcagtgcttctacgaggacatcgctcagggcaccaagtgcaccctgggagttccagtgattactggtggtcactgatatgtagattgtcgattagaagatcctgatggtaaagtgttatacaaagagattagtaacagttggtaatttttaccttacagcttccaaaaagggggacat

SEQ ID NO: 7 - DNA, 120 bp, Homo sapiens
acacataaaactgtatattttgattttaagttggagaagacccaccttttttcctagtgagaaccgagtagtgctcttacccagatggaatcgcctgtgtttcaattcacgaggctctga

SEQ ID NO: 8 - DNA, 367 bp, Homo sapiens
tttcttttgaaaagctttttctcagataaaagaaccaccacaactcgtgttggatcataactacgttttgagaattgatgcaccattgccactgtaatattgctgtcctctaattaattttaggtactgaagaacttaatattggcaacatttttaaatccttactcatacacttgttgggagggatgtacaatgcatattcccaaactgtggaaaggacacctttttttatttgtaaaggtggaaaactttggaactcattttgggctattcatgttaaatattcaacaccaatgatctactctgttcgcagttgtttatatctactcttcggacactaaacttgtattttggtcctttaaccttt

SEQ ID NO: 9 - DNA, 496 bp, Homo sapiens
aattcggcacgaggtgatatgtttaattttaacattttaattggctagctcttgcccttatatgactttaatgtctgtgagtcattcccagcttaaattaacaattgttagtattagtctcacacataagtgccatacattttatcctcatggatgtgatgcactgaaaagttagttgctctccttttttcttttttttgtcgtgcatattttatttctgtagtttctggttagctaccctaaagtgatttaaaaatttagaatgctttgtgtttcctatttggtaatcttcattgacttttctttagttaatgagtattaaatggtgcatatcctgtagactatagggttacaatggttgcaccttataacttggattagatggcgattgaataggtttggtcccagtttatccgttggatccaagtggttccaaatttttggtttaaggcttccgcggtttttttttagcaccgttttccgggccttcccccaa

SEQ ID NO: 10 - DNA, 27 bp, Homo sapiens
cgcccatggagatcaccttcgagcttc

SEQ ID NO: 11 - DNA, 27 bp, Homo sapiens
cgcaagcttgaaatgcctacatgtaag

SEQ ID NO: 12 - DNA, 27 bp, Homo sapiens
cgcaagcttcagcaatattacagtggc

SEQ ID NO: 13 - DNA, 38 bp, Homo sapiens
cgcggatccgccatcatgggcggccgtcgcgggcgttg

SEQ ID NO: 14 - DNA, 27 bp, Homo sapiens
cgcggtaccgaaatgcctacatgtaag

SEQ ID NO: 15 - DNA, 27 bp, Homo sapiens
cgcggtacccagcaatattacagtggc

SEQ ID NO: 16 - DNA, 24 bp, Homo sapiens
tgacagagggactttccgagagga